(19) United States
(12) Patent Application Publication
(10) Pub. No.: US 2014/0105919 A1
(43) Pub. Date: Apr. 17, 2014

(54) SYNERGISTIC INHIBITION OF ERBB2/ERBB3 SIGNAL PATHWAYS IN THE TREATMENT OF CANCER

(71) Applicant: Institute for Cancer Research d/b/a The Research Institute of Fox Chase Cancer Center, Philadelphia, PA (US)

(72) Inventors: Matthew K. Robinson, Blue Bell, PA (US); Smitha Reddy, Melville, NY (US)

(73) Assignee: Institute for Cancer Research d/b/a The Research Institute of Fox Chase Cancer Center, Philadelphia, PA (US)

(21) Appl. No.: 14/089,851

(22) Filed: Nov. 26, 2013

Related U.S. Application Data
(63) Continuation of application No. PCT/US2012/039526, filed on May 25, 2012.
(60) Provisional application No. 61/491,463, filed on May 31, 2011.

(51) Int. Cl. A61K 39/395; A61K 31/5415; A61K 31/135; A61K 31/713

(52) U.S. Cl. CPC A61K 39/39558; A61K 31/713; A61K 31/5415; A61K 31/135
USPC 424/174.1; 435/375

(57) ABSTRACT

Methods for treating tumors comprising cells overexpressing ErbB2 or ErbB3 are provided, and comprise inhibiting the biologic activity of one or more of ErbB2 and ErbB3 in the cells and inhibiting the expression or the biologic activity of a constituent of a signal pathway connected with ErbB2 or ErbB3 signaling.

### CROSS REFERENCE TO RELATED APPLICATIONS

This application is a continuation of International Application No. PCT/US2012/039526, filed on May 25, 2012, which claims priority to U.S. Provisional Application No. 61/491,463, filed on May 31, 2011, the contents of each are incorporated by reference herein, in their entirety and for all purposes.

### REFERENCE TO A SEQUENCE LISTING

This application includes a Sequence Listing submitted electronically as a text file named ErbB2 ErbB3 Sequence Listing_ST25.txt, created on May 24, 2012, with a size of 128,516 bytes. The Sequence Listing is incorporated by reference herein.

### FIELD OF THE INVENTION

The invention relates generally to the field of cancer treatment. More particularly, the invention relates to combination therapies for treating cancer cells overexpressing ErbB2 and/or ErbB3, and especially for treating drug-resistant cancer cells.

### BACKGROUND OF THE INVENTION

Various publications, including patents, published applications, accession numbers, technical articles and scholarly articles are cited throughout the specification. Each of these citations is incorporated by reference herein, in its entirety and for all purposes.

ErbB2 (also referred to as HER2) and ErbB3 (also referred to as HER3) are members of the epidermal growth factor family of receptor tyrosine kinases (RTKs) and are important drivers of both tumor formation and progression. ErbB3 has also been linked to resistance to targeted therapies such as trastuzumab.

Both ErbB2 and ErbB3 are targets for various small molecule and biomolecule therapeutic agents. Yet, many cancers become resistant to such agents as the cancers advance. Given the importance of the ErbB2 and ErbB3 targets in cancer therapy, there is a need for therapeutic regimens that can slow or overcome the drug resistance phenotype, as well as a need for therapeutic regimens that can enhance the efficacy of established agents such as those that target ErbB2 and ErbB3.

### SUMMARY OF THE INVENTION

The invention features various methods for treating tumors that comprise cells overexpressing ErbB2 and/or ErbB3. The methods relate to a combination therapy in which two aspects of cell signaling are inhibited—inhibiting the expression or the biologic activity of one or more of ErbB2 and ErbB3, and inhibiting the expression or the biologic activity of constituents of a signal network that bears some relation to ErbB2 or ErbB3 signaling. These methods may be carried out in vivo, in vitro, or in situ, and if carried out in vivo, may be used in accordance with any subject such as a mammal and preferably a human being.

A method for treating a tumor that comprises cells overexpressing ErbB2 or ErbB3 comprises inhibiting the expression or the biologic activity of either or both of ErbB2 and ErbB3 and inhibiting the expression or the biologic activity of one or more of: a constituent of the Notch signaling pathway, non-limiting examples of which include the Delta-1 ligand (DLL1), radical fringe (RFNG), nemo-like kinase (NLK), or lin-7 homolog A (LIN-7A); a constituent of the p53 signaling pathway such as MAP kinase 14 (MAPK14), dual specificity phosphatase 8 (DUSP8), or homeodomain-interacting protein kinase 2 (HIPK2); Met receptor tyrosine kinase (MET RTK); heat shock protein 90 (HSP90); sterol-C4-methyl oxidase-like protein (SC4MOL); hormonally regulated neu-associated kinase (HUNK); a constituent of the translationally-controlled tumor protein 1 (TPT1) signaling pathway, non-limiting examples of which include TPT1, cysteine rich angiogenic inducer 61 (CYR61), Ras GTPase-activating-like protein 1 (IQGAP1), or angio-associated migratory cell protein (AAMP); 55 kDa hematopoietic progenitor kinase 1 interacting protein (HIP-55); protein phosphatase 1 catalytic subunit Beta isoform (PP1CB); thyroid hormone receptor interactor 10 (TRIP10); delta 3, delta 2-enoyl-CoA isomerase (DCI); transmembrane protein 5 (TMEM5); human immunodeficiency virus type I enhancer binding protein 2 (HIVEP2); or bagpipe homeoboxprotein homolog 1 (BAPX1) in the cells.

As a result of the combined inhibition of the primary target, ErbB2 and/or ErbB3, and one or more of the secondary targets, the level of cell death in the tumor is enhanced, preferably statistically significantly enhanced, relative to the level of cell death in a tumor of the same type in which only the expression or the biologic activity of either the primary or secondary target by itself was inhibited. The combined inhibition results in a synergistic therapeutic benefit. In some aspects, the combined inhibition of the primary target, ErbB2 and/or ErbB3, and one or more of the secondary targets induces cancerous cells to revert from a cancer phenotype to a substantially normal or healthy phenotype. Thus, the methods are useful for effectuating enhanced killing of tumor cells and/or effectuating reversion of the cells to a healthy phenotype. Enhanced killing may relate, in part, to a reversion of a drug resistance phenotype in the cells, or to an enhanced susceptibility to one or more of the agents used to effectuate the inhibition of the primary or secondary targets.

The tumor may be any tumor in which ErbB2 and/or ErbB3 is overexpressed or overamplified, or in which ErbB2 and/or ErbB3 signaling plays a role in some aspect of tumor pathology, including cell proliferation. Non-limiting examples of such tumors include tumors of the breast, tumors of the lung, tumors of the stomach, tumors of the head and neck, tumors of the colon, tumors of the ovary, tumors of the pancreas, tumors of the prostate gland, and tumors of the esophagus and gastric-esophageal junction.

Expression of a target protein can be inhibited by transforming a cell with a nucleic acid molecule that specifically hybridizes to the mRNA encoding the target protein and interferes with translation. Examples of such nucleic acid molecules include, but are not limited to, siRNA, shRNA, and antisense RNA.

Inhibition of the biologic activity of a target protein can be effectuated by contacting the cell with an agent, which can be a chemical compound, a biomolecule, or a composition thereof, that inhibits the biologic activity. Any agent known in the art can be used to inhibit the target protein. Preferred categories of biomolecules include antibodies and regulatory peptides. Preferred categories of chemical compounds that inhibit the biologic activity of TPT1 include antidepressants and antihistamines.

The invention also features methods treating a malignancy of the breast, lung, esophagus, pancreas, stomach, colon, prostate, or head and neck that comprises cells which overexpress ErbB2 and/or ErbB3. These methods also relate to a combination therapy in which two aspects of cell signaling are inhibited—inhibiting the expression or the biologic activity of one or more of ErbB2 and ErbB3, and inhibiting the expression or the biologic activity of constituents of a signal network that bears some relation to ErbB2 or ErbB3 signaling. Such methods are carried out in vivo, on any subject in need of such treatment, such as a laboratory animal (e.g., mouse, rat, rabbit, non-human primate) or a human being.

A method for treating a malignancy of the breast, lung, esophagus, pancreas, stomach, colon, prostate, or head and neck comprising cells which overexpress ErbB2 and/or ErbB3 comprises administering to a subject in need thereof an effective amount of an agent that inhibits the expression or the biologic activity of either or both of ErbB2 and ErbB3 in tumor cells and an effective amount of an agent that inhibits the expression or the biologic activity of one or more of: a constituent of the Notch signaling pathway such as the Delta-1 ligand, radical fringe (RFNG), nemo-like kinase (NLK), or lin-7 homolog A (LIN-7A); a constituent of the p53 signaling pathway such as MAP kinase 14 (MAPK14), dual specificity phosphatase 8 (DUSP8), or homeodomain-interacting protein kinase 2 (HIPK2); Met receptor tyrosine kinase (MET RTK); heat shock protein 90 (HSP90); sterol-C4-methyl oxidase-like protein (SC4MOL); hormonally regulated neu-associated kinase (HUNK); a constituent of the translationally-controlled tumor protein 1 (TPT1) signaling pathway such as TPT1, cysteine rich angiogenic inducer 61 (CYR61), Ras GTPase-activating-like protein 1 (IQGAP1), or angio-associated migratory cell protein (AAMP); 55 kDa hematopoietic progenitor kinase 1 interacting protein (HIP-55); protein phosphatase 1 catalytic subunit Beta isoform (PP1CB); thyroid hormone receptor interactor 10 (TRIP10); delta 3, delta 2-enoyl-CoA isomerase (DCI); transmembrane protein 5 (TMEM5); human immunodeficiency virus type I enhancer binding protein 2 (HIVEP2); or bagpipe homeoboxprotein homolog 1 (BAPX1).

The agent can be a chemical compound, a biomolecule, or a composition thereof, that inhibits expression of the gene encoding the target protein of interest, or that inhibits the biologic activity of the target protein of interest. Any agent known in the art can be used, including those described or exemplified in this specification. Administration can be according to any suitable method.

### BRIEF DESCRIPTION OF THE DRAWINGS

FIG. 1A-D shows the ErbB/Notch interaction network; FIG. 1A shows the top left quadrant of a composite image, FIG. 1B shows the top right quadrant, FIG. 1C shows the bottom left quadrant, and FIG. 1D shows the bottom right quadrant.

FIG. 2A shows distribution of siRNA hits as a function of intrinsic activity. FIG. 2B shows the dynamic range of the siRNA screen.

FIG. 3 shows that combining MM-111 with the gamma-secretase inhibitor Compound E improves cell killing over MM-111 alone.

FIG. 4 shows that MM-111 induces Notch Signaling as measured with a reporter construct.

FIG. 5 shows that MM-111 induces expression of endogenous Notch target genes.

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

The term “about” encompasses variations of plus or minus 25%, 20%, 15%, 10%, 5%, 1%, 0.5%, 0.25%, or 0.1% from the specified value.

Knockdown includes the reduced expression of a gene. For example, a knockdown typically includes at least about a 20% reduction in expression, preferably includes at least about a 40% reduction in expression, preferably includes at least about a 50% reduction in expression, and more preferably includes at least about a 75% reduction in expression, and in some aspects includes at least about an 80% to about an 85% reduction in expression, at least about an 85% to about a 90% reduction in expression, or about an 80% to about a 90% reduction in expression, and in some aspects includes a greater than 90% reduction in expression, or a greater than 95% reduction in expression.

Transforming includes the introduction of exogenous or heterologous nucleic acid molecules into the cell. Cells may be stably or transiently transformed.

Nucleic acid molecules include any chain of at least two nucleotides, which may be unmodified or modified RNA or DNA, hybrids of RNA and DNA, and may be single, double, or triple stranded.

Expression of a nucleic acid molecule or gene includes the biosynthesis of a gene product. Expression encompasses, but is not limited to, the transcription of a gene into RNA, the translation of RNA into a protein or polypeptide, and all naturally occurring post-transcriptional and post-translational modifications thereof.

Biomolecules include proteins, polypeptides, antibodies, nucleic acid molecules, lipids, monosaccharides, polysaccharides, and all fragments, analogs, homologs, conjugates, and derivatives thereof.

Inhibiting or interfering includes reducing, decreasing, blocking, preventing, delaying, inactivating, desensitizing, stopping, knocking down (e.g., knockdown), and/or downregulating the biologic activity or expression of a molecule or pathway of interest.

It has been observed in accordance with the invention that inhibition of the expression of particular constituents of signal networks interconnected with ErbB2 and ErbB3 signaling in tumors can synergize with inhibition of ErbB2 and ErbB3 signaling, with the result of enhanced tumoricidal activity observed in tumors treated with this combined inhibition relative to tumors treated with either type of inhibition by itself. It has also been observed that inhibition of the expression of certain signal network constituents induces tumor cells to revert to a substantially normal, healthy phenotype. It has also been observed that inhibition of the expression of certain signal network constituents enhances susceptibility in tumor cells resistant to certain therapeutic agents. Signal networks previously identified as intertwined with ErbB signaling as well as signal networks not previously understood as having any relationship to ErbB signaling were characterized. Accordingly, the invention features various methods for treating tumors comprising cells overexpressing ErbB2 and/or ErbB3. Generally, the methods comprise inhibiting the expression or the biologic activity of either or both of ErbB2 and ErbB3 in cells overexpressing ErbB2 and/or ErbB3 and inhibiting the expression or the biologic activity of a constituent of an ErbB family-related signal pathway/network. Such constituents and ErbB family-related signal pathways are described and/or categorized in more detail below. The methods may be carried out in vivo, in vitro, or in situ.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of a constituent of the Notch signaling pathway in the cells. The constituent of the Notch signaling pathway may be, for example, one or more of the Delta-1 ligand, radical fringe (RFNG), nemo-like kinase (NLK), or lin-7 homolog A (LIN-7A). In preferred aspects, inhibiting both the expression or the biologic activity of ErbB2 and/or ErbB3 and the expression or the biologic activity of the Notch signaling pathway constituent in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of Met receptor tyrosine kinase (MET RTK) in the cells. In preferred aspects, inhibiting both the expression or the biologic activity of ErbB2 and/or ErbB3 and the expression or the biologic activity of MET RTK in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of heat shock protein 90 (HSP90) in the cells. In preferred aspects, inhibiting both the expression or the biologic activity of ErbB2 and/or ErbB3 and the expression or the biologic activity of HSP90 in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of sterol-C4-methyl oxidase-like protein (SC4MOL) in the cells. In preferred aspects, inhibiting both the expression or the biologic activity of ErbB2 and/or ErbB3 and the expression or the biologic activity of SC4MOL in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of hormonally regulated neu-associated kinase (HUNK) in the cells. In preferred aspects, inhibiting both the expression or the biologic activity of ErbB2 and/or ErbB3 and the expression or the biologic activity of HUNK in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of a constituent of the p53 signaling pathway in the cells. The constituent of the p53 signaling pathway may be, for example, one or more of MAP kinase 14 (MAPK14), dual specificity phosphatase 8 (DUSP8), or homeodomain-interacting protein kinase 2 (HIPK2). In preferred aspects, inhibiting both the expression or the biologic activity of ErbB2 and/or ErbB3 and the expression or the biologic activity of the p53 signaling pathway constituent in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In preferred aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of a constituent of the translationally-controlled tumor protein 1 (TPT1) signaling pathway in the cells. The constituent of the TPT1 signaling pathway may be, for example, one or more of TPT1, cysteine rich angiogenic inducer 61 (CYR61), Ras GTPase-activating-like protein 1 (IQGAP1), or angio-associated migratory cell protein (AAMP). In some preferred aspects, inhibiting both the expression or the biologic activity of ErbB2 and/or ErbB3 and the expression or the biologic activity of the TPT1 signaling pathway constituent in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling. In some preferred aspects, inhibiting both the expression or the biologic activity of ErbB2 and/or ErbB3 and the expression or the biologic activity of the TPT1 signaling pathway constituent in the cells induces the cells to revert from a cancer phenotype to a substantially normal or healthy phenotype.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of 55 kDa hematopoietic progenitor kinase 1 interacting protein (HIP-55) in the cells. HIP-55 is an adapter protein that is believed to link receptor tyrosine kinases to the cell cytoskeleton, and is believed to be involved in ErbB signaling. In preferred aspects, inhibiting both the expression or the biologic activity of ErbB2 and/or ErbB3 and the expression or the biologic activity of HIP-55 in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of protein phosphatase 1 catalytic subunit Beta isoform (PP1CB) in the cells. PP1CB is believed to be involved in actin cytoskeleton regulation. In preferred aspects, inhibiting both the expression or the biologic activity of ErbB2 and/or ErbB3 and the expression or the biologic activity of PP1CB in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of thyroid hormone receptor interactor 10 (TRIP10) in the cells. TRIP10 is an adapter protein and is believed to play a role in epidermal growth factor receptor internalization, and is believed to be involved with cytoskeleton regulation. In preferred aspects, inhibiting both the expression or the biologic activity of ErbB2 and/or ErbB3 and the expression or the biologic activity of TRIP10 in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of delta 3, delta 2-enoyl-CoA isomerase (DCI) in the cells. DCI is believed to play a role in fatty acid and sterol synthesis, and may be linked to SC4MOL. In preferred aspects, inhibiting both the expression or the biologic activity of ErbB2 and/or ErbB3 and the expression or the biologic activity of DCI in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of transmembrane protein 5 (TMEM5) in the cells. TMEM5 is a novel cell surface molecule, and is believed to be a potential drug target for cancer cells. In preferred aspects, inhibiting both the expression or the biologic activity of ErbB2 and/or ErbB3 and the expression or the biologic activity of TMEM5 in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of human immunodeficiency virus type I enhancer binding protein 2 (HIVEP2) in the cells. HIVEP2 is a DNA binding protein. In preferred aspects, inhibiting both the expression or the biologic activity of ErbB2 and/or ErbB3 and the expression or the biologic activity of HIVEP2 in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In some aspects, a method for treating a tumor comprising cells overexpressing ErbB2 or ErbB3 comprises inhibiting the expression or the biologic activity of either or both of ErbB2 and ErbB3 in the cells and inhibiting the expression or the biologic activity of bagpipe homeoboxprotein homolog 1 (BAPX1) in the cells. BAPX1 is a DNA binding protein. In preferred aspects, inhibiting both the expression or the biologic activity of ErbB2 and/or ErbB3 and the expression or the biologic activity of BAPX1 in the cells enhances the level of cell death in the tumor relative to the level of cell death in a tumor of the same type in which only the expression or biologic activity or ErbB2 and/or ErbB3 was inhibited. Cell death may be enhanced in tumors resistant to agents that inhibit ErbB2 or ErbB3 signaling.

In any of the methods, the expression of one or more of the target molecules (for example, ErbB2, ErbB3, Delta-1 ligand, RFNG, NLK, LIN-7A, HSP90, MET RTK, SC4MOL, DCI, HUNK, MAPK14, DUSP8, HIPK2, TPT1, CYR61, IQGAP1, AAMP, HIP-55, PP1CB, TRIP-10, TMEM5, HIVEP2, BAPX1 and other signal network constituents) can be inhibited, for example, by transforming tumor cells with a nucleic acid molecule that interferes with the expression of the gene encoding the target molecule in the cell, including the gene encoding ErbB2, ErbB3, Delta-1 ligand, RFNG, NLK, LIN-7A, HSP90, MET RTK, SC4MOL, DCI, HUNK, MAPK14, DUSP8, HIPK2, TPT1, CYR61, IQGAP1, AAMP, HIP-55, PP1CB, TRIP-10, TMEM5, HIVEP2, or BAPX1. Gene expression can be inhibited, for example, through the use of a variety of post-transcriptional gene silencing (RNA silencing) techniques.

RNA interference (RNAi) is a mechanism of post-transcriptional gene silencing mediated by double-stranded RNA (dsRNA), which is distinct from antisense and ribozyme-based approaches. RNA interference may be effectuated, for example, by administering a nucleic acid (e.g., dsRNA) that hybridizes under stringent conditions to the gene encoding the target molecule of interest, thereby attenuating its expression. RNA interference provides shRNA or siRNA that comprise multiple sequences that target one or more regions of the target gene. dsRNA molecules (shRNA or siRNA) are believed to direct sequence-specific degradation of mRNA in cells of various types after first undergoing processing by an RNase III-like enzyme called DICER into smaller dsRNA molecules comprised of two 21 nucleotide (nt) strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

Viral vectors or DNA vectors encode short hairpin RNA (shRNA) which are processed in the cell cytoplasm to short interfering RNA (siRNA). In general, a short, interfering RNA (siRNA) comprises an RNA duplex that is preferably approximately 19 basepairs long and optionally further comprises one or two single-stranded overhangs or loops. A siRNA may comprise two RNA strands hybridized together, or may alternatively comprise a single RNA strand that includes a self-hybridizing portion. siRNAs may include one or more free strand ends, which may include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In aspects in which perfect complementarity is not achieved, it is generally preferred that any mismatches be located at or near the siRNA termini.

siRNAs have been shown to downregulate gene expression when transferred into mammalian cells by such methods as transfection, electroporation, cationic liposome-mediated transfection, or microinjection, or when expressed in cells via any of a variety of plasmid-based approaches. The siRNA may consist of two individual nucleic acid strands or of a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is believed that intracellular processing (e.g., by DICER) of a variety of different precursors results in production of siRNA capable of effectively mediating gene silencing. Generally, it is preferred to target exons rather than introns, and it may also be preferable to select sequences complementary to regions within the 3′ portion of the target transcript. Generally it is preferred to select sequences that contain approximately equimolar ratio of the different nucleotides and to avoid stretches in which a single residue is repeated multiple times.

siRNAs may thus comprise RNA molecules having a double-stranded region approximately 19 nucleotides in length with 1-2 nucleotide 3′ overhangs on each strand, resulting in a total length of between approximately 21 and 23 nucleotides. siRNAs also include various RNA structures that may be processed in vivo to generate such molecules. Such structures include RNA strands containing two complementary elements that hybridize to one another to form a stem, a loop, and optionally an overhang, preferably a 3′ overhang. Preferably, the stem is approximately 19 by long, the loop is about 1-20, more preferably about 4-10, and most preferably about 6-8 nt long and/or the overhang is about 1-20, and more preferably about 2-15 nt long. In certain aspects, the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may, but need not comprise a plurality of U residues, e.g., between 1 and 5 U residues. Classical siRNAs as described above trigger degradation of mRNAs to which they are targeted, thereby also reducing the rate of protein synthesis. In addition to siRNAs that act via the classical pathway, certain siRNAs that bind to the 3′ UTR of a template transcript may inhibit expression of a protein encoded by the template transcript by a mechanism related to but distinct from classic RNA interference, e.g., by reducing translation of the transcript rather than decreasing its stability. Such RNAs are referred to as microRNAs (miRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop. Endogenous RNAs of this type have been identified in a number of organisms including mammals, suggesting that this mechanism of post-transcriptional gene silencing may be widespread. MicroRNAs have been shown to block translation of target transcripts containing target sites.

siRNAs such as naturally occurring or artificial (i.e., designed by humans) mRNAs that bind within the 3′ UTR (or elsewhere in a target transcript) and inhibit translation may tolerate a larger number of mismatches in the siRNA/template duplex, and particularly may tolerate mismatches within the central region of the duplex. In fact, there is evidence that some mismatches may be desirable or required as naturally occurring stRNAs frequently exhibit such mismatches as do mRNAs that have been shown to inhibit translation in vitro. For example, when hybridized with the target transcript such siRNAs frequently include two stretches of perfect complementarity separated by a region of mismatch. A variety of structures are possible. For example, the mRNA may include multiple areas of nonidentity (mismatch). The areas of nonidentity (mismatch) need not be symmetrical in the sense that both the target and the mRNA include nonpaired nucleotides. Typically the stretches of perfect complementarity are at least 5 nucleotides in length, e.g., 6, 7, or more nucleotides in length, while the regions of mismatch may be, for example, 1, 2, 3, or 4 nucleotides in length.

Hairpin structures designed to mimic siRNAs and mRNA precursors are processed intracellularly into molecules capable of reducing or inhibiting expression of target transcripts. These hairpin structures, which are based on classical siRNAs consisting of two RNA strands forming a 19 by duplex structure are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus, a diverse set of RNA molecules containing duplex structures is able to mediate silencing through various mechanisms. Any such RNA, one portion of which binds to a target transcript and reduces its expression, whether by triggering degradation, by inhibiting translation, or by other means, may be considered an siRNA, and any structure that generates such an siRNA (i.e., serves as a precursor to the RNA) is useful.

A further method of RNA interference is the use of short hairpin RNAs (shRNA). A plasmid containing a DNA sequence encoding for a particular desired siRNA sequence is delivered into a target cell via transfection or virally-mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for times periods longer than several days. In some cases, though, this may be preferable and desired. In cases where longer periods of protein inhibition are necessary, shRNA mediated inhibition is preferable. The use of shRNA is preferred for some aspects of the invention. Typically, siRNA-encoding vectors are constructs comprising a promoter, a sequence of the target gene to be silenced in the sense orientation, a spacer, the antisense of the target gene sequence, and a terminator.

Inhibition of the expression of the target signal constituent can also be effectuated by other means that are known and readily practiced in the art. For example, antisense nucleic acids can be used. Antisense RNA transcripts have a base sequence complementary to part or all of any other RNA transcript in the same cell. Such transcripts modulate gene expression through a variety of mechanisms including the modulation of RNA splicing, the modulation of RNA transport and the modulation of the translation of mRNA. Accordingly, in certain aspects, inhibition of the expression of the target signal protein in a cell can be accomplished by expressing an antisense nucleic acid molecule in the cell.

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically, they are oligonucleotides that range from 15 to 35 nucleotides in length but may range from 10 up to approximately 50 nucleotides in length. Binding typically reduces or inhibits the expression of the target nucleic acid, such as the gene encoding the target signal protein. For example, antisense oligonucleotides may block transcription when bound to genomic DNA, inhibit translation when bound to mRNA, and/or lead to degradation of the nucleic acid. Inhibition of the expression of the target signal protein can be achieved by the administration of antisense nucleic acids comprising sequences complementary to those of the mRNA that encodes the target signal protein.

Antisense oligonucleotides can be synthesized with a base sequence that is complementary to a portion of any RNA transcript in the cell. Antisense oligonucleotides can modulate gene expression through a variety of mechanisms including the modulation of RNA splicing, the modulation of RNA transport and the modulation of the translation of mRNA. Various properties of antisense oligonucleotides including stability, toxicity, tissue distribution, and cellular uptake and binding affinity may be altered through chemical modifications including (i) replacement of the phosphodiester backbone (e.g., peptide nucleic acid, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C).

Inhibition of the target signal molecule can also be effectuated by use of ribozymes. Certain nucleic acid molecules referred to as ribozymes or deoxyribozymes have been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave to any RNA molecule, thereby increasing its rate of degradation.

In some aspects, the cells can be specifically transformed with transcription-silencing nucleic acids such as shRNA or siRNA, or can be transformed with vectors encoding such nucleic acids such that the cell expresses the inhibitory nucleic acid molecules. Transformation of the cells can be carried out according to any means suitable in the art.

A cell can be transformed with such nucleic acid molecules according to any means available in the art such as those describe or exemplified herein. It is preferred that cells are stably transformed with a vector comprising a nucleic acid sequence encoding such regulatory nucleic acid molecules, although transiently transformations are suitable. Any vector suitable for transformation of the particular cell of interest can be used. In preferred embodiments, the vector is a viral vector. In some embodiments, the viral vector is a lentivirus vector.

In some aspects, the methods comprise inhibiting the expression or the biologic activity of either or both of ErbB2 and ErbB3 in the cells and inhibiting the expression or the biologic activity of one or more of Delta-1 ligand, RFNG, NLK, LIN-7A, HSP90, MET RTK, SC4MOL, DCI, HUNK, MAPK14, DUSP8, HIPK2, TPT1, CYR61, IQGAP1, AAMP, HIP-55, PP1CB, TRIP-10, TMEM5, HIVEP2, and BAPX1. Two, three, four, or more such targets may be used in a combination therapy method. Moreover, it is not necessary that the selected targets share the same signal pathway. For example, a target may be selected from the Notch signal pathway and from the TPT1 signal pathway.

In any of the methods, the biologic activity of one or more of the target proteins (for example, ErbB2, ErbB3, Delta-1 ligand, RFNG, NLK, LIN-7A, HSP90, MET RTK, SC4MOL, DCI, HUNK, MAPK14, DUSP8, HIPK2, TPT1, CYR61, IQGAP1, AAMP, HIP-55, PP1CB, TRIP-10, TMEM5, HIVEP2, BAPX1 and other signal network constituents) can be inhibited, for example, by contacting tumor cells with a compound, biomolecule, or composition of a compound or a biomolecule that inhibits the biologic activity of the target protein in the cell. Preferred biomolecules include peptide inhibitors and antibodies.

Non-limiting examples of antibodies that inhibit ErbB2 and ErbB3 include trastuzumab, pertuzumab, U3-1287, and M-121. In some aspects, antibodies have dual specificity for ErbB2 and ErbB3. Dual-specific antibodies include MM-111, manufactured by Merrimack Pharmaceuticals, Inc., and the antibodies described in U.S. Pat. Nos. 7,332,580 and 7,332,585. An anti-EGFR antibody, panitumumab, may be used in some aspects. The antibodies ficlatuzumab and rilotumumab bind the ligand for c-MET, and block signaling through the MET receptor, and may be used in some aspects.

The small molecule tyrosine kinase inhibitor lapatinib may be used in some aspects. Non-limiting categories of small molecules that downregulate TPT1 include antihistamines and antidepressants. Examples of agents in such categories include the FDA-approved drugs promethazine (an antihistamine), and the chemically related anti-depressants thioridazine and sertraline. Examples of inhibitors of Met that may be used in some aspects include, but are not limited to Onartuzumab (MetMab), Tivantinib (ARQ197), JNJ-38877605, PF-04217903, SGX-523, Crizotinib (PF-02341066), and Cabozantinib (XL-184).

TPT1 was identified as regulator of the TSC-Rheb pathway (Hsu et al. (2007) Nature 445:785-788). TSC-Rheb pathway is implicated in diseases such as tuberous sclerosis complex (renal) and lymphangioleiomyomatosis (lung). TSC (tuberous sclerosis complex) is a direct regulator of mTOR (mammalian Target Of Rapamycin). mTOR is instrumental for integrating various stress/growth stimuli and generating an appropriate cell growth response. Thus, rapamycin and its analogs (temsirolimu, everolimus/RAD001, and deforolimus) may be used as a way of disrupting TPT1-dependent effects.

The methods thus relate to a combination treatment, targeting ErbB2 and/or ErbB3 and targeting a constituent of a signal network related to ErbB2 or ErbB3 signaling. For the combination, ErbB2 and/or ErbB3 may be inhibited before the constituent of the signal network is inhibited, may be inhibited substantially at the same time as that the constituent of the signal network is inhibited, or may be inhibited after the constituent of the signal network is inhibited.

The methods may be used to treat any cancer (or tumor type) in which ErbB2 and/or ErbB3 is overexpressed, or in which ErbB2 and/or ErbB3 signaling mediates development, progression, pathology, or resistance to one or more chemotherapeutic agents. Non-limiting examples of such cancers include breast cancer, head and neck cancer, colon cancer, prostate cancer, ovarian cancer, pancreatic cancer, lung cancer, esophageal cancer, gastric cancer, and cancer of the gastric/esophageal junction among others.

The invention also features methods for treating a malignancy of the breast, head and neck, colon, ovary, prostate, pancreas, esophagus, lung, or stomach comprising cells overexpressing either or both of ErbB2 and ErbB3. In general, such methods comprise administering to a subject in need thereof an effective amount of agent that inhibits the biologic activity of one or more of ErbB2 and ErbB3, and an effective amount agent the inhibits the expression of the gene encoding one or more of the Delta-1 ligand, RFNG, NLK, LIN-7A, HSP90, MET RTK, SC4MOL, DCI, HUNK, MAPK14, DUSP8, HIPK2, TPT1, CYR61, IQGAP1, AAMP, HIP-55, PP1CB, TRIP-10, TMEM5, HIVEP2, and/or BAPX1, or an effective amount of an agent that inhibits the biologic activity of one or more of the Delta-1 ligand, RFNG, NLK, LIN-7A, HSP90, MET RTK, SC4MOL, DCI, HUNK, MAPK14, DUSP8, HIPK2, TPT1, CYR61, IQGAP1, AAMP, HIP-55, PP1CB, TRIP-10, TMEM5, HIVEP2, or BAPX1 protein.

In some aspects, the agent that inhibits the expression of the gene encoding one or more of the Delta-1 ligand, RFNG, NLK, LIN-7A, HSP90, MET RTK, SC4MOL, DCI, HUNK, MAPK14, DUSP8, HIPK2, TPT1, CYR61, IQGAP1, AAMP, HIP-55, PP1CB, TRIP-10, TMEM5, HIVEP2, and/or BAPX1 is a nucleic acid molecule that interferes with the expression of the gene. After administration, said nucleic acid molecule transforms a malignant cell of the breast, head and neck, colon, ovary, prostate, pancreas, esophagus, lung, or stomach that is overexpressing either or both of ErbB2 or ErbB3, and then interferes with the expression of the RFNG gene, NLK gene, LIN-7A gene, HSP90 gene, MET RTK gene, SC4MOL gene, DCI gene, HUNK gene, MAPK14 gene, DUSP8 gene, HIPK2 gene, TPT1 gene, CYR61 gene, IQGAP1 gene, AAMP gene, HIP-55 gene, PP1CB gene, TRIP-10 gene, TMEM5 gene, HIVEP2 gene, or BAPX1 gene in the transformed cell. The nucleic acid molecule may be administered to or specifically targeted to the cells of interest, or at least to an area proximal to the cells of interest. Transformation of the cells may be facilitated according to any suitable technique.

In some aspects, the agent that inhibits the biologic activity of one or more of the Delta-1 ligand, RFNG, NLK, LIN-7A, HSP90, MET RTK, SC4MOL, DCI, HUNK, MAPK14, DUSP8, HIPK2, TPT1, CYR61, IQGAP1, AAMP, HIP-55, PP1CB, TRIP-10, TMEM5, HIVEP2, or BAPX1 is a compound, biomolecule, or composition comprising a compound or biomolecule that inhibits the biologic activity of the target protein in the cell. The compound or biomolecule may be specific to the particular target molecule, or may be a non-specific inhibitor. Preferred biomolecules include peptide inhibitors and antibodies. Non-limiting categories of small molecules that inhibit TPT1 include antihistamines and antidepressants. Examples of agents in such categories include the FDA-approved drugs promethazine (an antihistamine), and the chemically related anti-depressants thioridazine and sertraline. (Amson R et al. (2012) Nature Med. 18:91-9).

The agents may be administered according to any technique suitable in the art. The subject to which the agents are administered may be any animal, preferably mammals, and including laboratory animals (e.g., rodents such as mice, rabbits, and rats), companion animals (e.g. cats and dogs), farm animals (e.g., horses, cows, pigs, sheep), and non-human primates. Human beings are preferred subjects.

The methods of treatment include a combination therapy, by targeting both ErbB2 and/or ErbB3 and a constituent of a signal network related to ErbB2 or ErbB3 signaling. For the combination, the agent for inhibiting the biologic activity of ErbB2 and/or ErbB3 may be administered to the subject before the agent that inhibits the expression or the biologic activity of the constituent of the signal network is administered to the subject, may be administered to the subject substantially at the same time as the agent that inhibits the expression or the biologic activity of the constituent of the signal network is administered to the subject, or may be administered to the subject after the agent that inhibits the expression or the biologic activity of the constituent of the signal network is administered to the subject.

The following examples are provided to describe the invention in greater detail. They are intended to illustrate, not to limit, the invention.

### EXAMPLE 1

### Pathways Previously Identified to Interact with Inhibitors of ErbB Signaling

A antibody with dual specificity for ErbB2 and ErbB3 was used in combination with a siRNA synthetic lethal screen to identify additive or synergistic treatments. The antibody bears the name MM-111, and is under clinical evaluation by Merrimack Pharmaceuticals, Inc. The antibody MM-111 is a derivative of the single chain antibodies described in U.S. Pat. Nos. 7,332,580 and 7,332,585.

A large-scale (^{˜}6800) siRNA synthetic lethal screen was undertaken in the breast cancer cell line MDA-MB-361/DYT2, a subcloned line of the ErbB2-positive, ErbB3-positive breast cancer cell line MDA-MB-361 (ATCC cat #HTB-27) to identify pathways that are additive or synergistic with MM-111 treatment. The Dharmacon® “Druggable siRNA library” was used for the screen to bias hits toward proteins that can be targeted with either small molecule or biologic-based inhibitors. Each of the ^{˜}6800 genes represented in the library was targeted by a pool of 4 siRNAs in the screen. The siRNA library was aliquoted across 86 master plates with 8 positive and 8 negative controls on each plate. Each master plate was then transfected into 4 plates of MDA-MB361 breast cancer cells (3000 cells/well) and duplicate plates were treated with either 2 μM MM-111 (approximately IC₂₀) or appropriate vehicle control. Cells were then assayed for viability using CellTiter-Blue®(Promega, cat #G808B) as recommended by the manufacturer. Positive (AllStars, Qiagen cat #1027299) and negative (GL2, Dharmacon cat #D-001100-01-20) controls for cell death were included on each plate to control for plate-to-plate and day-to-day variations across the screen. A viability score was assigned, in which values greater than 1 indicate cell viability and values lesser than 1 indicate cell death.

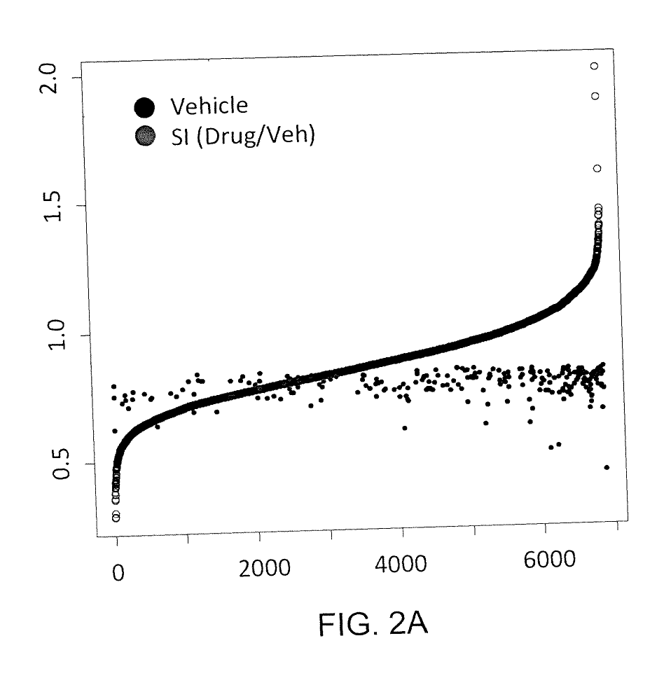

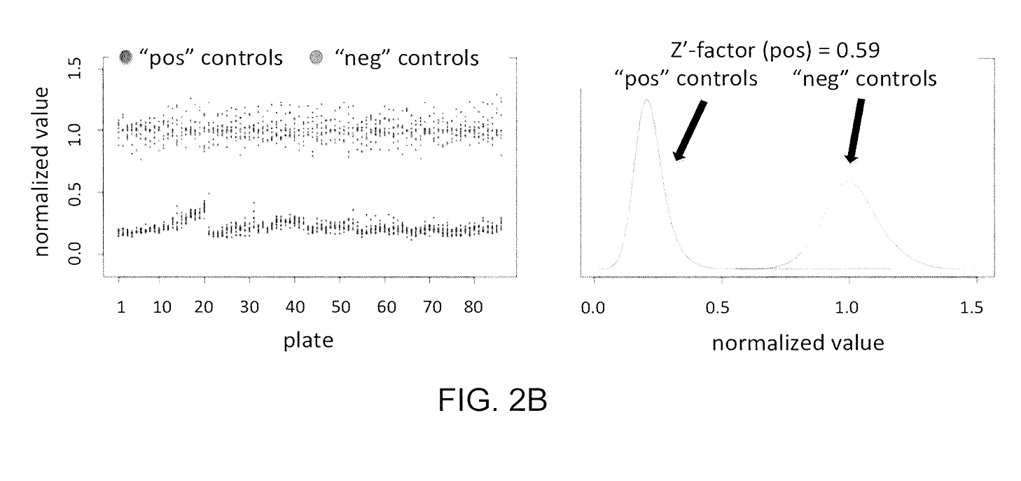

The siRNA assay was robust. As shown in FIG. 2A, siRNAs were ranked in order of intrinsic activity (open circles). Hits, defined as ratio of combination treated to vehicle treated of <0.85 and false detection rate <20%, are shown in the solid dots. Hits were found across the entire spectrum of intrinsic activities. The left side of the curve shows siRNAs that induce cell death, and the right side of the curve shows siRNAs that do not induce cell death, and at the end of the spectrum, induce cells to grow.

The dynamic range of the assay is shown in FIG. 2B. Each plate for the siRNA screen was carried out in duplicate. Data in figure are representative of one replicate. The left panel shows normalized values of 8 positive (bottom) and 8 negative (top) controls on each plate of screen, representing a range of cell viabilities across each plate of the screen. The right panel depicts a Z-score (0.59) determined from positive (left curve) and negative (right curve) control data.

Based on the statistical criteria of having a false-detection rate (FDR) of ≦20% and a viability ratio of 0.85 (MM-111 treated:vehicle treated), 238 genes (approximately 3.5% of the total) were identified that, when targeted in combination with MM-111, resulted in increased therapeutic efficacy over either component alone. Of these 238 unique genes, 74 had FDRs of <10% with 19 of those having an FDR of <5%. The results are shown in Table 1. These hits include both previously identified interactions with the ErbB family and novel interactions.

TABLE 1
Synthetic Lethal siRNA Screen Using Pooled siRNAs with MM-111.

| Gene Symbol | Gene Accession | Vehicle Avg | Drug Avg | Ratio |
|---|---|---|---|---|
| AAMP | NM_001087 | 0.950 | 0.654 | 0.689 |
| BAPX1 | NM_001189 | 1.168 | 0.803 | 0.688 |
| CYR61 | NM_001554 | 0.736 | 0.506 | 0.688 |
| DCI | NM_001919 | 0.807 | 0.563 | 0.697 |
| DLL1 | NM_005618 | 0.975 | 0.735 | 0.754 |
| DUSP8 | NM_004420 | 0.795 | 0.600 | 0.754 |
| HIP-55 | NM_001014436 | 1.053 | 0.550 | 0.522 |
| HIVEP2 | NM_006734 | 1.168 | 0.829 | 0.710 |
| HIPK2 | NM_022740 | 0.826 | 0.668 | 0.810 |
| HUNK | NM_014586 | 0.589 | 0.437 | 0.742 |
| IQGAP1 | NM_003870 | 1.122 | 0.892 | 0.795 |
| LIN-7A | NM_004664 | 1.378 | 1.025 | 0.743 |
| MAPK14 | NM_139013 | 0.880 | 0.629 | 0.715 |
| MET | NM_000245 | 1.027 | 0.795 | 0.774 |
| NLK | NM_016231 | 0.800 | 0.634 | 0.793 |
| NTRK3 | NM_002530 | 1.173 | 0.932 | 0.795 |
| PPP1CB | NM_002709 | 0.571 | 0.416 | 0.729 |
| RFNG | NM_002917 | 0.947 | 0.582 | 0.615 |
| SC4MOL | NM_001017369 | 0.876 | 0.679 | 0.774 |
| TMEM5 | NM_014254 | 1.080 | 0.783 | 0.725 |
| TPT1 | NM_003295 | 0.841 | 0.630 | 0.749 |
| TRA1 | NM_003299 | 1.130 | 0.854 | 0.756 |
| TRP10 | NM)004240 | 1.284 | 0.850 | 0.662 |

Table 1. The sensitization of MDA-MB361 breast cancer cells, as measured by cell titer blue viability assay, to selected siRNAs in the presence of siRNA only (vehicle) or siRNA and the HER2/HER3 bispecific antibody, MM-111. Displayed genes represent screen hits with a false detection rate (FDR) of <5% (bolded) in the initial screen, suspected biological interaction with ERBB network, or combination of the two.

Among the pathways previously identified to interact with ERb signaling inhibitors:

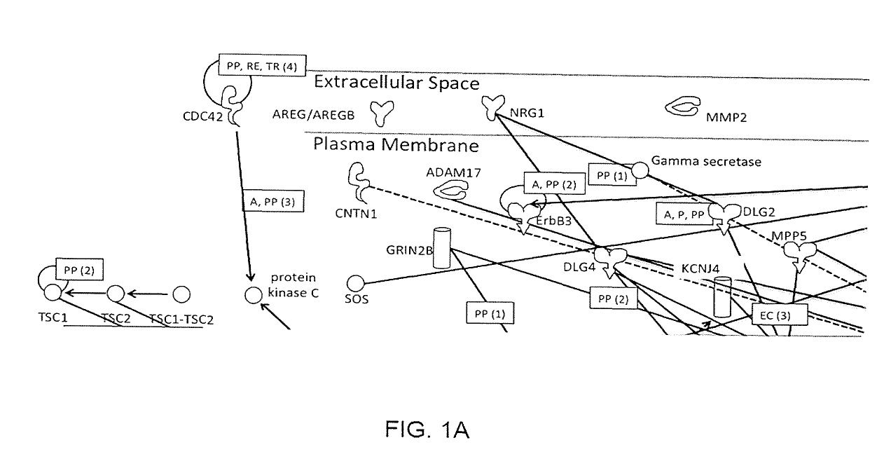

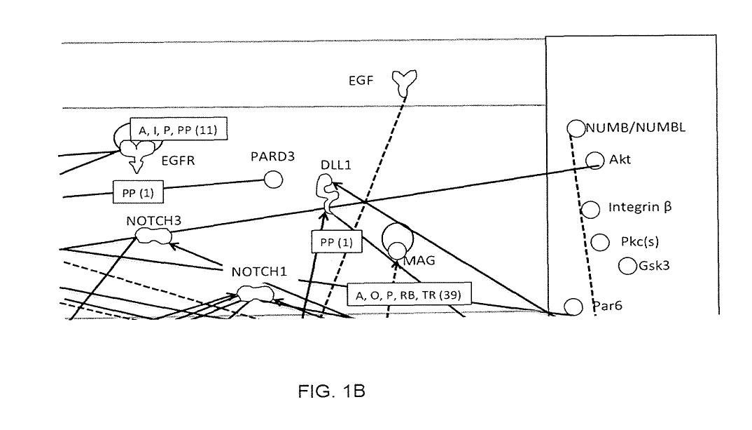

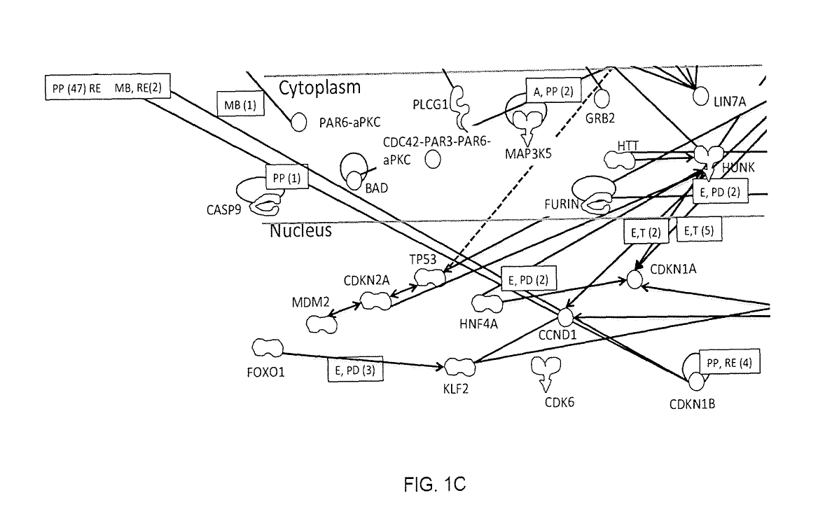

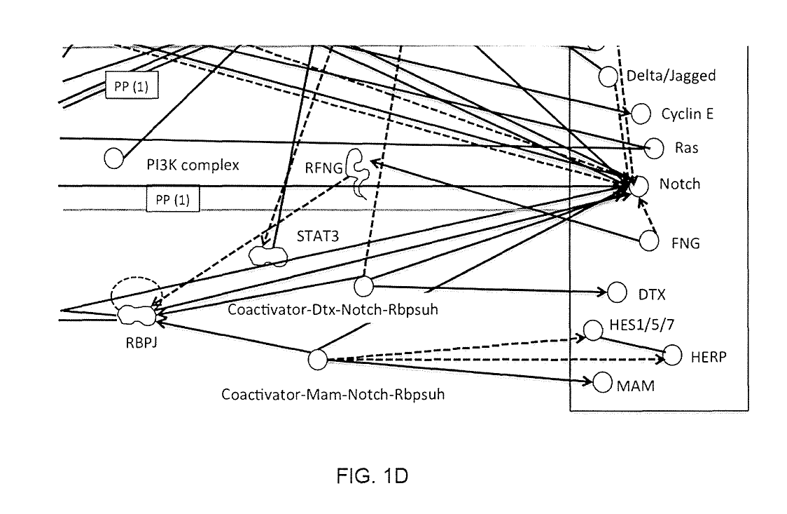

1. The Notch pathway. Four components of the Notch signaling pathway were identified. Notch receptor signaling is known to interact with ErbB signaling in organisms ranging from C. elegans to humans. The interaction is detailed in FIG. 1.

The experiments identified Delta-1 (a Notch ligand that activates signaling), RFNG (a critical component that positively regulates Notch expression on the cell surface), and NLK (a kinase downstream of Notch) and LIN-7A (an adaptor protein that interacts with ErbB receptors and is believed to be necessary for known cross-talk between Notch and ErbB signaling). There are known pharmacologic inhibitors of Notch signaling (MK0752 and 804929097) that are being tested clinically alone and in combination with other agents (e.g., cetuximab+RO49829097 in the setting of colorectal cancer). These results suggest that combination therapies with Notch inhibitors and MM-111 will have at least additive effects against tumors that utilize both pathways.

2. The Met signaling pathway. This signaling pathway interacts with HERVErbB3 to result in resistance to cetuximab and other EGFR-targeted therapies. The MET RTK was isolated in the screen as well as a large number of genes associated with MET signaling.

3. Ubiquitin pathway. The screen identified components of the ubiquitination and degradation pathway. There are known interactions between ErbB family members and the proteosome inhibitor bortezomib that resulted in clinical trials of bortezomib in combination with trastuzumab and in combination with cetuximab.

4. SC4MOL. SC4MOL was isolated through the screen. SC4MOL was identified in a similar screen using cetuximab and other EGFR-targeted agents.

5. HUNK. The hormonally regulated neu-associated kinase (HUNK) was identified in the screen. HUNK is a kinase known to be involved in integrating estrogen receptor and ErbB2 signaling.

Summary. The screen was designed to identify interactions between MM-111 and other potential drug targets. Although 238 genes were identified, only 127 appear to be expressed in the cell line used for the experiment. These include 45 with an FDR <10% with 14 of those having an FDR <5%. What this screen was able to accomplish was to limit the sphere of pathways that should be investigated in combination with MM-111. Additional validation experiments using both siRNA and pharmacologic inhibitors will be carried out to validate primary hits, and are currently underway.

### EXAMPLE 2

### Pathways Newly Identified to Interact with Inhibitors of ErbB Signaling

The siRNA screen identified new pathways that are affected by inhibitors of ErbB signaling. Among these newly identified pathways are the following:

1. TPT1 node. This network node is comprised of TPT1/TCTP (NM_{—}003295), CYR61 (NM_{—}001554), AAMP (NM_{—}001087), and IQGAP1 (NM_{—}003870) (FIG. 1). Proteins in this node are linked to cell migration and angiogenesis. Inhibitors of TCTP expression are known in the literature (Tuynder et al. (2004) PNAS 101:15364). It is believed that ErbB inhibitors (e.g., MM-111) will synergize with inhibitors of TCTP (e.g., small molecule inhibitors described in Tuynder et al.; and regulatory nucleic acid approaches). The cellular functions ascribed to TCTP in the literature suggest that this effect will be enhanced in combination with taxane or other microtubule destabilizing agent. Alternatively, inhibitors of other members of this node may have similar activity.

2. MAPK14/DUSP8 node. Interactions with MM-111 were identified within the ErbB/p53 (TP53) interaction network. These include the MAPK kinase MAPK14 (NM_{—}001315) and dual specificity phosphatase DUSP8 (NM_{—}004420). Both of these proteins have been linked to stress response pathway signaling. The nuclear ser/thr kinase HIPK2 was also identified. It is involved in transcriptional regulation in response to genotoxic stress. These proteins represent potential targets for small molecule inhibitors or other therapeutics (e.g., siRNAs) that could be used in conjunction with ErbB inhibitors.

3. NTRK3 (NM_{—}002530). NTRK3 is a member of the neurotrophic tyrosine receptor kinase (NTRK) family. Upon neurotrophin binding, it phosphorylates itself and members of the MAPK pathway. Mutations in this gene have been associated with medulloblastomas, secretory breast carcinomas and other cancers.

4. TMEM5 (NM_{—}014254). TMEM5 is a novel Type H transmembrane protein with unknown function. Antibody-based inhibitors of this protein could be used in conjunction with ErbB inhibitors to achieve a synergistic effect.

### EXAMPLE 3

### Validation Experiments

Subsets of targets that met criteria of being expressed in the cells and having an FDR of <5% or being expressed in the cells, having an FDR <20%, and having a known connection to ErbB signaling, were validated in a secondary screen in which MB361/DYT2 breast cancer cells were treated with siRNA molecules from the Dharmacon® siGENOME library (Dharmacon, Inc.). The sequences are shown in Table 2. Parallel cells were treated with the siRNA only (vehicle) or both the siRNA and the ErbB2/ErbB3 bispecific antibody MM-111, and sensitization of the cells was measured by the CellTiter-Blue® (Promega) viability assay. The results of this screen are shown in Table 3.

TABLE 2
Dharmacon siGENOME siRNA Screen Validation Sequences

| Gene | | |
|---|---|---|
| Target | siRNA 1 | siRNA 2 |
| DLL1 | GCACGGAUCUCGAGAACAG (SEQ ID NO: 1) | CAUAAGCCCUGCAAGAAUG (SEQ ID NO: 17) |
| RFNG | GUCAAGUUCUGGUUUGCUA (SEQ ID NO: 2) | GAACGUGGCUGGAGGCUUC (SEQ ID NO: 18) |
| NLK | GGUGGAAGAUAAUGUACUA (SEQ ID NO: 3) | GGUGUUGUCUGGUCAGUAA (SEQ ID NO: 19) |
| MET | GAAGAUCAGUUUCCUAAUU (SEQ ID NO: 4) | CCAGAGACAUGUAUGAUAA (SEQ ID NO: 20) |
| SC4MOL | GAACAGACUCUCAGUAUAA (SEQ ID NO: 5) | GAAGAUACUUGGCACUAUU (SEQ ID NO: 21) |
| HUNK | UCACUCAGCUCCUUGAUAU (SEQ ID NO: 6) | GAUAGAGAAUUUGCUACUA (SEQ ID NO: 22) |
| MAPK14 | CAAGGUCUCUGGAGGAAUU (SEQ ID NO: 7) | GUCAGAAGCUUACAGAUGA (SEQ ID NO: 23) |
| TPT1 | AGAUGUUCUCCGACAUCUA (SEQ ID NO: 8) | GGUUGUGCCUGGAGGUGGA (SEQ ID NO: 24) |
| CYR61 | GGGCAGACCCUGUGAAUAU (SEQ ID NO: 9) | GGCCAGAAAUGUAUUGUUC (SEQ ID NO: 25) |
| AAMP | CCACAAAGCGAAAGUAUUU (SEQ ID NO: 10) | CCAAAGGCCUGACCGUUAA (SEQ ID NO: 26) |
| HIP-55 | GCACAUUGACCACCACAUU (SEQ ID NO: 11) | ACUCUGGACUGCCCAAAUU (SEQ ID NO: 27) |
| PPP1CB | CACCAGACCUGCAAUCUAU (SEQ ID NO: 12) | GAAGUUCGAGGCUUAUGUA (SEQ ID NO: 28) |
| DCI | CCAAAGACUCCAUCCAGAA (SEQ ID NO: 13) | GAACUUCGUCAGCUUCAUC (SEQ ID NO: 29) |
| HIVEP2 | GAAAGAAACCUGCUGAGUA (SEQ ID NO: 14) | GAAGAUACAUGACCACAAU (SEQ ID NO: 30) |
| BAPX1 | UAAAGGUGCUGGUGCGCGA (SEQ ID NO: 15) | AGGCGAUCCUCAACAAGAA (SEQ ID NO: 31) |
| TMEM5 | UCAGUGGCCUUUAGGAGUA (SEQ ID NO: 16) | ACAGAAUGCUAUCGAAUCU (SEQ ID NO: 32) |
| Gene | | |
| Target | siRNA 3 | siRNA 4 |
| DLL1 | CAUAGCAACUGAGGUGUAA (SEQ ID NO: 33) | GCACGCACCCUGCCACAAU (SEQ ID NO: 49) |
| RFNG | ACGUGGAUGAUGACAAUUA (SEQ ID NO: 34) | CCACACGGUUUAAGUCUAU (SEQ ID NO: 50) |
| NLK | CAACUGUGUUCUAAAGAUU (SEQ ID NO: 35) | GGACGAAGAAUAUUGUUUC (SEQ ID NO: 51) |
| MET | GAACAGAAUCACUGACAUA (SEQ ID NO: 36) | GAAACUGUAUGCUGGAUGA (SEQ ID NO: 52) |
| SC4MOL | GAACUUCAUUGGAAACUAU (SEQ ID NO: 37) | GGUGACCAUUCGUUUAUUA (SEQ ID NO: 53) |
| HUNK | GAAGAUGGUAGACAAAGAA (SEQ ID NO: 38) | AUAGAGAAUUUGCUACUAG (SEQ ID NO: 54) |
| MAPK14 | GUCCAUCAUUCAUGCGAAA (SEQ ID NO: 39) | CUACAGAGAACUGCGGUUA (SEQ ID NO: 55) |
| TPT1 | UCUACAAGAUCCGGGAGAU (SEQ ID NO: 40) | CGAAGGUACCGAAAGCACA (SEQ ID NO: 56) |
| CYR61 | GGUCAAAGUUACCGGGCAG (SEQ ID NO: 41) | GCAGCAAGACCAAGAAAUC (SEQ ID NO: 57) |
| AAMP | UGACAAAGCCUUCGUAUGG (SEQ ID NO: 42) | GGAAAGUCCCGAAUGGUGA (SEQ ID NO: 58) |
| HIP-55 | CCAAUGCCGUGUCUGAGAU (SEQ ID NO: 43) | CGACACAGAGAUCUCCUUU (SEQ ID NO: 59) |
| PPP1CB | GAUCGUGGUGUUUCCUUUA (SEQ ID NO: 44) | GAACGUGGACAGCCUCAUC (SEQ ID NO: 60) |
| DCI | GGGUCGCUGUGAUGAAAUU (SEQ ID NO: 45) | GCUGGUGGCUUCUGUGCGA (SEQ ID NO: 61) |
| HIVEP2 | GGACACAGCUCUAGGACAA (SEQ ID NO: 46) | CAACCAACAUCCUAUAUGA (SEQ ID NO: 62) |
| BAPX1 | GCUUUAACCACCAGCGCUA (SEQ ID NO: 47) | ACUAUUACCCGUACUACUG (SEQ ID NO: 63) |
| TMEM5 | CCGUUGAUGUGAAUAAUGU (SEQ ID NO: 48) | GAGGCAAGUUGGUCAAUGC (SEQ ID NO: 64) |

TABLE 3
Dharmacon siGENOME Synthetic Lethal siRNA Screen
for MM-111: Validation Data Grouped by Function.

| | | siRNA | | | |
|---|---|---|---|---|---|
| Notch Pathway | Accession No. | Sequence | Vehicle | Drug | Ratio |
| DLL1 | NM_005618 | SEQ ID | 0.987 | 0.812 | 0.823 |
| | | NO: 17 | | | |
| NLK | NM_016231 | SEQ ID | 0.993 | 0.991 | 0.776 |
| | | NO: 19 | | | |
| RFNG** | NM_002917 | SEQ ID | 1.331 | 1.250 | 0.939 |
| | | NO: 50 | | | |

AKT Activation and Signaling

| | | | | | |
|---|---|---|---|---|---|
| AAMP | NM_001087 | SEQ ID | 0.973 | 0.821 | 0.844 |
| | | NO: 26 | | | |
| CYR61 | NM_001554 | SEQ ID | 0.988 | 0.846 | 0.856 |
| | | NO: 57 | | | |
| IQGAP1 | NM_003870 | * | | | N/D |
| TPT1 | NM_003295 | SEQ ID | 03917 | 0.775 | 0.845 |
| | | NO: 24 | | | |

Cholesterol/Fatty Acid Synthesis

| | | | | | |
|---|---|---|---|---|---|
| DCI | NM_001919 | SEQ ID | 0.823 | 0.627 | 0.762 |
| | | NO: 45 | | | |
| SCMOL | NM_001017369 | SEQ ID | 1.089 | 0.955 | 0.877 |
| | | NO: 21 | | | |

* sequence unavailable.
**not validated.

### EXAMPLE 4

### RT-PCR Evaluation of Degree of Knockdown

The degree of mRNA knockdown by pooled siRNA in cells maintained and treated as described in the Examples above was assessed by RT-PCR. MDA-MB361/DYT, BT474, HR6, SK-OV-3, SK-BR-3 cells were assessed. Cells were transfected in 6-well plates using Dharmafect1 transfection reagent and Opti-MEM with 250 nM pooled siRNA and a cell density of 120,000 cells per well. Media was changed after 24 hours and cells were collected following a 72 hour incubation period. Total RNA was extracted from cells using the RNeasy kit (Qiagen). GL2 and AllStar (Qiagen) were used as negative and positive controls, respectively, to ensure transfection efficiency. Total RNA was reverse transcribed to cDNA, and real time PCR was performed, using ABI primer/probe sets, on cDNAs to detect relative expression of NLK (context sequence TCATAAACAGCCATCTCTTCCTGTA, SEQ ID NO:65), DLL1 (context sequence CTGCACAGAGCCGATCTGCCTGCCT, SEQ ID NO:66), and RFNG (context sequence CATTGAGTCCGGGCGCAAGTGGTTT, SEQ ID NO:67). HPRT1 (context sequence GGTTAAGGTTGCAAGCTTGCTGGTG, SEQ ID NO:68) and IPO8 (context sequence GGGGAATTGATCAGTGCATTCCACT, SEQ ID NO:69) were used as loading controls.

The percent knockdown is shown in Table 4. Values are the percent remaining relative to untransfected cells after normalizing all samples to IPO8 and HPRT1 internal controls. DL11 was determined to be below the detectable threshold in both control and transfected cells for the SK-OV-3 and SK-BR-3 cell lines.

TABLE 4
Degree of mRNA knockdown by pooled siRNAs.

| | MDA-MB361 | BT474 | HR6 | SK-OV-3 | SK-BR-3 |
|---|---|---|---|---|---|
| NLK | 13.99% | 17.81% | 17.66% | 15.81% | 40.22% |
| RFNG | 13.08% | 6.69% | 10.38% | 17.71% | 13.24% |
| DLL1 | 41.01% | 44.36% | 40.40% | N/A | N/A |

### EXAMPLE 5

### Cell Sensitization to MM-111 and Gamma-Secretase Inhibitor

Inhibition of the Notch pathway was carried out with a compound. Breast cancer cells (BT474 cells) were plated in 96-well plates in DMEM/F12 media with 1% FBS and incubated in a humidified CO₂ incubator at 37° C. for 24 hours prior to drug treatment. The media was replaced with DMEM/F12 media with 1% FBS containing DMSO, 1 μM Compound E (Santa Cruz, SC-221433), 2 μM MM-111 (Merrimack Pharmaceuticals), or combined Compound E and MM-111. Cells were incubated in the presence of drug for 72 hrs. CellTiter-Blue® (Promega) was used to measure cell viability according to the standard protocol. The total incubation time in the presence of CellTiter-Blue® was 4 hours, and fluorescence was measured using a Perkin Elmer Envision Plate Reader.

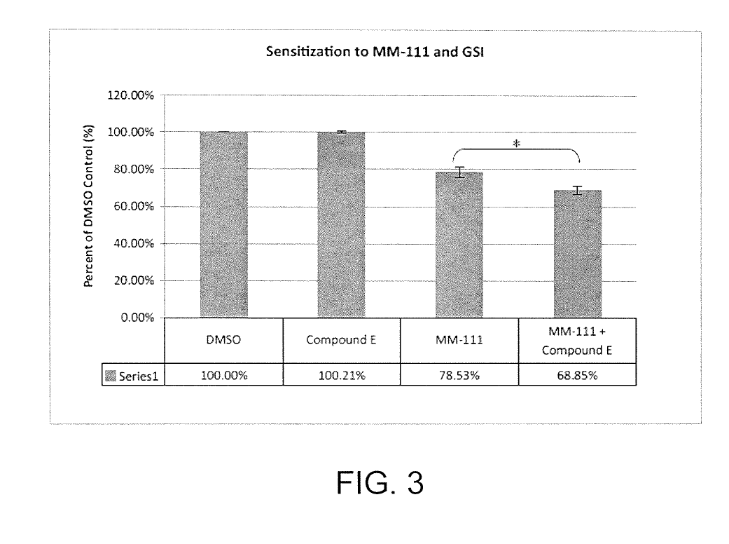

As shown in FIG. 3, combining MM-111 with the gamma-secretase inhibitor (GSI) Compound E improves cell killing. The ERBB2-overexpressing breast cancer cell line BT474 was treated with vehicle (DMSO), Compound E (1 μM), MM-111 (2 μM), or MM-111 and Compound E. Compound E had no effect on viability, and MM-111 alone showed a reduction in viability. Consistent with results from siRNA screen, inhibiting the Notch pathway by blocking GSI activity with Compound E in combination with MM-111 trended toward improved efficacy as compared to MM-111 alone (*p=0.063).

### EXAMPLE 6

### Notch-Dependent Activation of -CBF-1 Reporter

BT-474 cells were co-transfected with CBF1::firefly luciferase and pRL-TK (renilla luciferase) as an internal control for transfection efficiency. Cells were then treated with MM-111, MM-111 and Compound E, or Rituximab (control antibody), and the ratio of firefly/renilla expression was measured using the Dual-Luciferase Assay (Promega) according to the manufacturer's instructions.

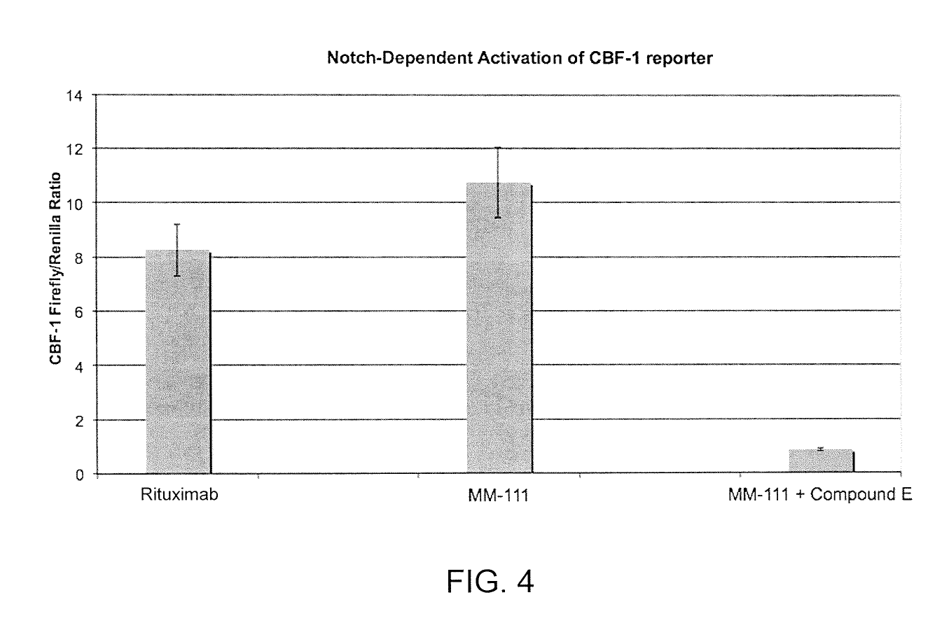

As shown in FIG. 4, MM-111 Induces Notch Signaling. ERBB2-dependent signaling is believed to suppress Notch activation. Treatment of BT-474 cells with MM-111 blocks signaling through ERBB2/ERBB3 heterodimer and induces Notch activation as compared to a negative control antibody (Rituximab) when measured by expression of a CBF1 reporter construct. Consistent with activation of the reporter being Notch-dependent, the activity is blocked by the gamma secretase inhibitor Compound E.

### EXAMPLE 7

### Notch-Dependent Activation of Endogenous Transcriptional Targets

MB-361/DYT2 cells were left untreated (NT) or treated with DMSO (vehicle for Compound E), rituximab (control antibody), Compound E, MM-111, or MM-111 and Compound E. Total RNA was extracted from cells using the RNeasy kit (Qiagen). Total RNA was reverse transcribed to cDNA, and real time PCR was performed, using ABI primer/probe sets, on cDNAs to detect relative expression of the validated Notch target genes HES1 (ABI Assay ID Hs00172878_m1), HES5 (ABI Assay ID Hs01387463_g1), or HEY1 (ABI Assay ID Hs01114113_m1).

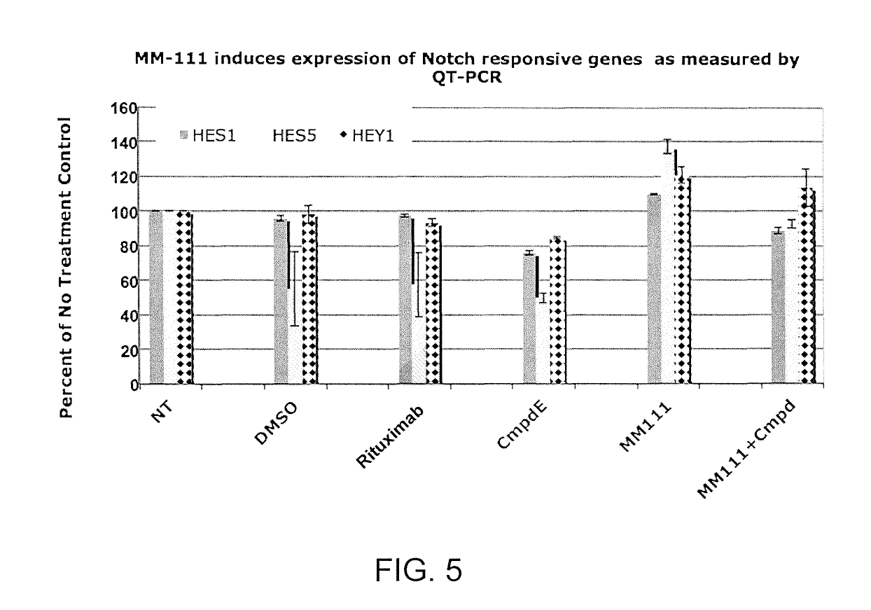

As shown in FIG. 5, MM-111 induced Notch Signaling. ERBB2-dependent signaling suppressed Notch activation. Treatment of MB361/DYT2 cells with MM-111 blocked signaling through ERBB2/ERBB3 heterodimer and leads to increased expression of Notch target genes HES1, HES5, and HEY1 as compared to untreated cells and cells treated with either DMSO or a negative control antibody (Rituximab). Consistent with Compound E blocking Notch signaling, expression of HES1, HES5, and HEY1 was reduced in Compound E-treated cells. The combination of MM-111 and Compound E blocks MM-111-dependent increase in expression of HES1, HES5, and HEY1, as compared to an actin control (ABI Assay ID Hs99999903_m1).

### EXAMPLE 8

### Combination Therapy for Treatment of Prostate Cancer

It is believed that additional signaling pathways combine with ErbB2/ErbB3 signaling in prostate cancer to drive formation and progression. Developing strategies to effectively exploit the currently available HER-targeted therapies for the treatment of androgen independent prostate cancer will require identification of these cooperative pathways, and the synthetic lethality screen with the antibody MM-111 described above was designed to identify essential components of those pathways. One candidate target protein is TPT1. TPT1 is a highly conserved protein that plays critical roles in multiple cellular functions including regulating cell shape and migration. One mechanism by which it is proposed to do this is through regulation of the mTOR pathway by directly interacting with Rheb, a Ras-family member, involved in the pathway. Signaling through mTOR and through ErbB2/ErbB3 are both known to support growth of prostate cancer cells in the absence of androgen, a hallmark of advanced disease. Additionally, TPT1 expression directly impacts on the survival of prostate cancer cells. Therefore, it is believed that inhibition of TPT1 expression or activity will synergize with inhibition of ErbB to enhance the treatment of prostate cancer.

To investigate this possibility, in vitro studies will be undertaken to: 1) establish the effect of combining TPT1 and ErbB-targeted inhibitors on the growth of androgen independent prostate cancer. Based on TPT1's role in regulating microtubule dynamics, these experiments will be carried out in the presence and absence of the microtubule stabilizing agent docetaxel that is within the standard of care for androgen independent prostate cancer; and, 2) investigate the mechanism by which TPT1 inhibition enhances the cell killing activity of ErbB-targeted agents. Specific emphasis will be given to studying the impact of combinations on AR stability and androgen independent activation of the androgen receptor. It is envisioned that data generated from these studies will support future testing of the combinations in both animals and ultimately patients.

Experiments will be carried out in the HER2+/HER3+ rogen-dependent-Prostate Cancer cell lines CWR22 and LNCaP as well as their androgen independent derivatives CWR22Rv1 (ATCC #CRL-2505) and C4-2 (and C4-2B). The impact of pharmacologic and siRNA-based inhibitors of TPT1 will be analyzed alone and in combination with the ErbB-inhibitors MM111, lapatinib, trastuzumab and pertuzumab, as well as the rapamycin analog RAD-001. This will be done in the presence and absence of docetaxel. The impact of individual and combination therapies on these cell lines will be evaluated using standard colony forming and MTS-based assays. The method of Chou/Talalay will be used to determine combination indices (CI) as a measure of drug/drug synergy. Stability, as well as transcriptional readouts, of androgen receptor activity in both androgen-independent and androgen-dependent prostate cancer will be evaluated in the context of treatment combinations to begin investigating the mechanism of crosstalk between the two pathways.

The invention is not limited to the embodiments described and exemplified above, but is capable of variation and modification within the scope of the appended claims.

We claim:

1. A method for treating a tumor comprising cells overexpressing ErbB2 or ErbB3, the method comprising inhibiting the biologic activity of one or more of ErbB2 and ErbB3 in the cells and inhibiting the biologic activity of or the expression of a gene encoding a constituent of the translationally-controlled tumor protein 1 (TPT1) signaling pathway selected from the group consisting of cysteine rich angiogenic inducer 61 (CYR61), Ras GTPase-activating-like protein 1 (IQGAP1), angio-associated migratory cell protein (AAMP), and TPT1 in the cells, wherein inhibiting the expression or the biologic activity of the constituent in the cells enhances the level of cell death in the tumor induced by inhibiting the biologic activity of ErbB2 or ErbB3 relative to the level of cell death in a tumor of the same type in which the expression or the biologic activity of the constituent was not inhibited.

2. The method of claim 1, wherein the tumor is a tumor of the breast, tumor of the lung, tumor of the stomach, tumor of the head and neck, tumor of the colon, tumor of the ovary, tumor of the prostate, tumor of the pancreas, a tumor of the esophagus, or a tumor of the gastric-esophageal junction.

3. The method of claim 1, wherein inhibiting the expression of a gene encoding a constituent of the TPT1 signaling pathway comprises transforming the cells with a nucleic acid molecule that interferes with the expression of the gene.

4. The method of claim 3, wherein the constituent is TPT1 and the nucleic acid molecule is a siRNA that specifically hybridizes under stringent conditions to mRNA encoding the TPT1.

5. The method of claim 3, wherein the constituent is CYR61 and the nucleic acid molecule is a siRNA that specifically hybridizes under stringent conditions to mRNA encoding the CYR61.

6. The method of claim 3, wherein the constituent is IQGAP1 and the nucleic acid molecule is a siRNA that specifically hybridizes under stringent conditions to mRNA encoding the IQGAP1.

7. The method of claim 3, wherein the constituent is AAMP and the nucleic acid molecule is a siRNA that specifically hybridizes under stringent conditions to mRNA encoding the AAMP.

8. The method of claim 1, wherein inhibiting the biologic activity of a constituent of the TPT1 signaling pathway comprises contacting the cells with an effective amount of a compound that inhibits the biologic activity of the constituent.

9. The method of claim 8, wherein the constituent is TPT1 and the compound is an antidepressant or an antihistamine.

10. The method of claim 9, wherein the antihistamine is promethazine.

11. The method of claim 9, wherein the antidepressant is thioridazine or sertraline.

12. The method of claim 1, wherein inhibiting the biologic activity of one or more of ErbB2 and ErbB3 comprises contacting the cells with an effective amount of an antibody that specifically binds to and inhibits the biologic activity of ErbB2, an antibody that specifically binds to and inhibits the biologic activity of ErbB3, or an antibody that specifically binds to and inhibits the biologic activity of a heterodimer of ErbB2 and ErbB3.

13. A method for treating a malignancy of the breast, lung, esophagus, pancreas, stomach, colon, ovary, prostate, gastric-esophageal junction, or head and neck comprising cells overexpressing ErbB2 or ErbB3, comprising transforming a malignant cell of the breast, lung, esophagus, pancreas, stomach, colon, prostate, or head and neck comprising cells overexpressing ErbB2 or ErbB3 in a subject in need thereof with an effective amount of a nucleic acid molecule that interferes with the expression of a gene encoding one or more of TPT1, CYR61, IQGAP1, and AAMP, and administering to the subject an effective amount of an antibody that specifically binds to and inhibits the biologic activity of ErbB2, an antibody that specifically binds to and inhibits the biologic activity of ErbB3, or an antibody that specifically binds to and inhibits the biologic activity of a heterodimer of ErbB2 and ErbB3.

14. The method of claim 13, wherein the nucleic acid molecule that interferes with the expression of the gene encoding TPT1 is a siRNA that specifically hybridizes under stringent conditions with TPT1 mRNA.

15. The method of claim 13, wherein the nucleic acid molecule that interferes with the expression of the gene encoding CYR61 is a siRNA that specifically hybridizes under stringent conditions with CYR61 mRNA.

16. The method of claim 13, wherein the nucleic acid molecule that interferes with the expression of the gene encoding IQGAP1 is a siRNA that specifically hybridizes under stringent conditions with IQGAP1 mRNA.

17. The method of claim 13, wherein the nucleic acid molecule that interferes with the expression of the gene encoding AAMP is a siRNA that specifically hybridizes under stringent conditions with AAMP mRNA.

18. The method of claim 13, wherein the subject is a human being.

19. A method for treating a malignancy of the breast, lung, esophagus, pancreas, stomach, colon, ovary, prostate, gastric-esophageal junction, or head and neck comprising cells overexpressing ErbB2 or ErbB3, comprising administering to a subject in need thereof an effective amount of an agent that inhibits the biologic activity of one or more of TPT1, CYR61, IQGAP1, and AAMP, and administering to the subject an effective amount of an antibody that specifically binds to and inhibits the biologic activity of ErbB2, an antibody that specifically binds to and inhibits the biologic activity of ErbB3, or an antibody that specifically binds to and inhibits the biologic activity of a heterodimer of ErbB2 and ErbB3.

20. The method of claim 19, wherein agent that inhibits the biologic activity of TPT1 comprises an antidepressant or an antihistamine.

21. The method of claim 20, wherein the antihistamine is promethazine.

22. The method of claim 20, wherein the antidepressant is thioridazine or sertraline.


SEQUENCE LISTING (92 sequences)

SEQ ID NO: 1 - RNA, 19 nt, Homo sapiens
gcacggaucucgagaacag

SEQ ID NO: 2 - RNA, 19 nt, Homo sapiens
gucaaguucugguuugcua

SEQ ID NO: 3 - RNA, 19 nt, Homo sapiens
gguggaagauaauguacua

SEQ ID NO: 4 - RNA, 19 nt, Homo sapiens
gaagaucaguuuccuaauu

SEQ ID NO: 5 - RNA, 19 nt, Homo sapiens
gaacagacucucaguauaa

SEQ ID NO: 6 - RNA, 19 nt, Homo sapiens
ucacucagcuccuugauau

SEQ ID NO: 7 - RNA, 19 nt, Homo sapiens
caaggucucuggaggaauu

SEQ ID NO: 8 - RNA, 19 nt, Homo sapiens
agauguucuccgacaucua

SEQ ID NO: 9 - RNA, 19 nt, Homo sapiens
gggcagacccugugaauau

SEQ ID NO: 10 - RNA, 19 nt, Homo sapiens
ccacaaagcgaaaguauuu

SEQ ID NO: 11 - RNA, 19 nt, Homo sapiens
gcacauugaccaccacauu

SEQ ID NO: 12 - RNA, 19 nt, Homo sapiens
caccagaccugcaaucuau

SEQ ID NO: 13 - RNA, 19 nt, Homo sapiens
ccaaagacuccauccagaa

SEQ ID NO: 14 - RNA, 19 nt, Homo sapiens
gaaagaaaccugcugagua

SEQ ID NO: 15 - RNA, 19 nt, Homo sapiens
uaaaggugcuggugcgcga

SEQ ID NO: 16 - RNA, 19 nt, Homo sapiens
ucaguggccuuuaggagua

SEQ ID NO: 17 - RNA, 19 nt, Homo sapiens
cauaagcccugcaagaaug

SEQ ID NO: 18 - RNA, 19 nt, Homo sapiens
gaacguggcuggaggcuuc

SEQ ID NO: 19 - RNA, 19 nt, Homo sapiens
gguguugucuggucaguaa

SEQ ID NO: 20 - RNA, 19 nt, Homo sapiens
ccagagacauguaugauaa

SEQ ID NO: 21 - RNA, 19 nt, Homo sapiens
gaagauacuuggcacuauu

SEQ ID NO: 22 - RNA, 19 nt, Homo sapiens
gauagagaauuugcuacua

SEQ ID NO: 23 - RNA, 19 nt, Homo sapiens
gucagaagcuuacagauga

SEQ ID NO: 24 - RNA, 19 nt, Homo sapiens
gguugugccuggaggugga

SEQ ID NO: 25 - RNA, 19 nt, Homo sapiens
ggccagaaauguauuguuc

SEQ ID NO: 26 - RNA, 19 nt, Homo sapiens
ccaaaggccugaccguuaa

SEQ ID NO: 27 - RNA, 19 nt, Homo sapiens
acucuggacugcccaaauu

SEQ ID NO: 28 - RNA, 19 nt, Homo sapiens
gaaguucgaggcuuaugua

SEQ ID NO: 29 - RNA, 19 nt, Homo sapiens
gaacuucgucagcuucauc

SEQ ID NO: 30 - RNA, 19 nt, Homo sapiens
gaagauacaugaccacaau

SEQ ID NO: 31 - RNA, 19 nt, Homo sapiens
aggcgauccucaacaagaa

SEQ ID NO: 32 - RNA, 19 nt, Homo sapiens
acagaaugcuaucgaaucu

SEQ ID NO: 33 - RNA, 19 nt, Homo sapiens
cauagcaacugagguguaa

SEQ ID NO: 34 - RNA, 19 nt, Homo sapiens
acguggaugaugacaauua

SEQ ID NO: 35 - RNA, 19 nt, Homo sapiens
caacuguguucuaaagauu

SEQ ID NO: 36 - RNA, 19 nt, Homo sapiens
gaacagaaucacugacaua

SEQ ID NO: 37 - RNA, 19 nt, Homo sapiens
gaacuucauuggaaacuau

SEQ ID NO: 38 - RNA, 19 nt, Homo sapiens
gaagaugguagacaaagaa

SEQ ID NO: 39 - RNA, 19 nt, Homo sapiens
guccaucauucaugcgaaa

SEQ ID NO: 40 - RNA, 19 nt, Homo sapiens
ucuacaagauccgggagau

SEQ ID NO: 41 - RNA, 19 nt, Homo sapiens
ggucaaaguuaccgggcag

SEQ ID NO: 42 - RNA, 19 nt, Homo sapiens
ugacaaagccuucguaugg

SEQ ID NO: 43 - RNA, 19 nt, Homo sapiens
ccaaugccgugucugagau

SEQ ID NO: 44 - RNA, 19 nt, Homo sapiens
gaucgugguguuuccuuua

SEQ ID NO: 45 - RNA, 19 nt, Homo sapiens
gggucgcugugaugaaauu

SEQ ID NO: 46 - RNA, 19 nt, Homo sapiens
ggacacagcucuaggacaa

SEQ ID NO: 47 - RNA, 19 nt, Homo sapiens
gcuuuaaccaccagcgcua

SEQ ID NO: 48 - RNA, 19 nt, Homo sapiens
ccguugaugugaauaaugu

SEQ ID NO: 49 - RNA, 19 nt, Homo sapiens
gcacgcacccugccacaau

SEQ ID NO: 50 - RNA, 19 nt, Homo sapiens
ccacacgguuuaagucuau

SEQ ID NO: 51 - RNA, 19 nt, Homo sapiens
ggacgaagaauauuguuuc

SEQ ID NO: 52 - RNA, 19 nt, Homo sapiens
gaaacuguaugcuggauga

SEQ ID NO: 53 - RNA, 19 nt, Homo sapiens
ggugaccauucguuuauua

SEQ ID NO: 54 - RNA, 19 nt, Homo sapiens
auagagaauuugcuacuag

SEQ ID NO: 55 - RNA, 19 nt, Homo sapiens
cuacagagaacugcgguua

SEQ ID NO: 56 - RNA, 19 nt, Homo sapiens
cgaagguaccgaaagcaca

SEQ ID NO: 57 - RNA, 19 nt, Homo sapiens
gcagcaagaccaagaaauc

SEQ ID NO: 58 - RNA, 19 nt, Homo sapiens
ggaaagucccgaaugguga

SEQ ID NO: 59 - RNA, 19 nt, Homo sapiens
cgacacagagaucuccuuu

SEQ ID NO: 60 - RNA, 19 nt, Homo sapiens
gaacguggacagccucauc

SEQ ID NO: 61 - RNA, 19 nt, Homo sapiens
gcugguggcuucugugcga

SEQ ID NO: 62 - RNA, 19 nt, Homo sapiens
caaccaacauccuauauga

SEQ ID NO: 63 - RNA, 19 nt, Homo sapiens
acuauuacccguacuacug

SEQ ID NO: 64 - RNA, 19 nt, Homo sapiens
gaggcaaguuggucaaugc

SEQ ID NO: 65 - DNA, 25 bp, Homo sapiens
tcataaacagccatctcttcctgta

SEQ ID NO: 66 - DNA, 25 bp, Homo sapiens
ctgcacagagccgatctgcctgcct

SEQ ID NO: 67 - DNA, 25 bp, Homo sapiens
cattgagtccgggcgcaagtggttt

SEQ ID NO: 68 - DNA, 25 bp, Homo sapiens
ggttaaggttgcaagcttgctggtg

SEQ ID NO: 69 - DNA, 25 bp, Homo sapiens
ggggaattgatcagtgcattccact

SEQ ID NO: 70 - DNA, 1859 bp, Homo sapiens
aggggctcgagttccgcgtcgtcgcgcagagctgactctgggaggcgtttgggcccagagaagtggatccgccgcttgcgccgcatggagtccgaatcggaaagcggggctgctgctgacacccccccactggagaccctaagcttccatggtgatgaagagattatcgaggtggtagaacttgatcccggtccgccggacccagatgacctggcccaggagatggaagatgtggactttgaggaagaagaggaggaagagggcaacgaagagggctgggttctagaaccccaggaaggggtggtcggcagcatggagggccccgacgatagcgaggtcacctttgcattgcactcagcatctgtgttttgtgtgagcctggaccccaagaccaataccttggcagtgaccgggggtgaagatgacaaagccttcgtatggcggctcagcgatggggagctgctctttgagtgtgcaggccataaagactctgtgacttgtgctggtttcagccatgactccactctagtggccacaggggacatgagtggcctcttgaaagtgtggcaggtggacactaaggaggaggtctggtcctttgaagcgggagacctggagtggatggagtggcatcctcgggcacctgtcctgttggcgggcacagctgacggcaacacctggatgtggaaagtcccgaatggtgactgcaagaccttccagggtcccaactgcccagccacctgtggccgagtcctccctgatgggaagagagctgtggtaggctatgaagatgggaccatcaggatttgggacctgaagcagggaagccctatccatgtactgaaagggactgagggtcaccagggcccactcacctgtgttgctgccaaccaggatggcagcttgatcctaactggctctgtggactgccaggccaagctggtcagtgccaccaccggcaaggtggtgggtgtttttagacctgagactgtggcctcccagcccagcctgggagaaggggaggagagtgagtccaactcggtggagtccttgggcttctgcagtgtgatgcccctggcagctgttggctacctggatgggaccttggccatctatgacctggctacgcagactcttaggcatcagtgtcagcaccagtcgggcatcgtgcagctgctgtgggaggcaggcactgccgtggtatatacctgcagcctggatggcatcgtgcgcctctgggacgcccggaccggccgcctgcttactgactaccggggccacacggctgagatcctggactttgccctcagcaaagatgcctccctggtggtgaccacgtcaggagaccacaaagcgaaagtattttgtgtccaaaggcctgaccgttaatggctgcagcccctgcctgtgtgtctggtgttgaggggacgaagggacccctgcccctgtctgccagcagaggcagtagggcacagagggaagaggagggtggggccctggatgactttccagcctcttcaactgacttgctcccctctccttttcttctctttagagacccagcccagggccctcccacccttgtccagacctggtgggcccttcagagggaggggtggacctgtttctctttcactttcatttgctggtgtgagccatggggtgtgtatttgtatgtggggagtaggtgtttgaggttcccgttctttcccttcccaagtctctgggggtggaaaggaggaagagatactagttaaagattttaaaaatgtaaataaaatatacttcccagaaaaaaaaaaaaaaaaaaaaaaaaaaaaaaaaaaaaaaaaaaaaaaaaaaaaaaaaaaaaaaaaaaa

SEQ ID NO: 71 - DNA, 2241 bp, Homo sapiens
ggcgctctctgtcccgctcggagctgctcggcgccccagctgcccgccccgccggccgctcctgcccgcggcgcagatggctgtgcgcggcgccaacaccttgacgtccttctccatccaggcgatcctcaacaagaaagaggagcgcggcgggctggccgcgccagaggggcgcccggcgcccgggggcacagcggcatcggtggccgcggctcccgctgtctgctgttggcggctctttggggagagggacgcgggcgcgttggggggcgccgaggactctctgctggcgtctcctgccggtaccagaacagctgcggggcggactgcggagagcccggaaggctgggactcggactccgcgctcagcgaggagaacgagagcaggcggcgctgcgcggacgcgcggggggccagcggggccggccttgcggggggatccttgagcctcggccagccggtctgtgagctggccgcttccaaagacctagaggaggaagccgcgggccggagcgacagcgagatgtccgccagcgtctcaggcgaccgcagcccaaggaccgaggacgacggtgttggccccagaggtgcacacgtgtccgcgctgtgcagcggggccggcggcgggggcggcagcgggccggcaggcgtcgcggaggaggaggaggagccggcggcgcccaagccacgcaagaagcgctcgcgggccgctttctcccacgcgcaggtcttcgagctggagcgccgctttaaccaccagcgctacctgtccgggcccgagcgcgcagacctggccgcgtcgctgaagctcaccgagacgcaggtgaaaatctggttccagaaccgtcgctacaagacaaagcgccggcagatggcagccgacctgctggcctcggcgcccgccgccaagaaggtggccgtaaaggtgctggtgcgcgacgaccagagacaatacctgcccggcgaagtgctgcggccaccctcgcttctgccactgcagccctcctactattacccgtactactgcctcccaggctgggcgctctccacctgcgcagctgccgcaggcacccagtgaacccgcttgggctgaggcagcgagtgattcccgcgctccggctccggaccggcgctgacagctgtaggctgtagcctgcacggggcgccccgccaaggaggcacctggaggtgaaacccagctccagctcccgttagccaggacttgtcccctggcagctgggctgagtctgccctgagggggcgcctttttctaatttgaacagaggcaccctatggcctaggggccctgatcgcccacctgcctggaagcccctgggctctatttattatcatgacaatgttggaattaaattttgattcgaatatgtctgcctgggggtggggttttccctgagcggcaactcctggagaccacatagcctgaatcctcagaatttcaggcctgctgggagctttctgcactaggccacactagttcatggtatccatgctaccaatctatgtgtatctacatatcttttatttttggaaattgcatttgtaaccaaggggtgcgaaaccctggcagtcccaggcagcaccaggccaggggttgatttgaaacgtgaaggattgggttttcaggccctctgctccacccctcctgtgtgtcagagctagggtgggggtgcccgattcgggtgctgaatgtaaggaggggagcctccaagtgtggtgcaagccgggggtctccacatcttccttctctgaagtccaggtacctgcacaagcaggaagcgcctgggagtcccggaaggaggagagcgcacacccaggcagccctctgcggaaactttccttggtttctttttatttgtgtaaaggaggttaagacgtgtcgcacttttcagttgtttgtattcaaatgacgattatttttctactcaatgtgaatatccctggccagcctttccacggcgcccaccgcagtgccgctgcctggccctcagtgtctaccttctgccctctgcgactccagtgctctggcccgggactcccctatccgcccctcacttacccttaaacaggtgatcccacctgtcttgtcaacctcgccgcttttcgcctccttaatggcactgtgcactcaactagagtattaactgtaaaaagatttgtgaagtttggaagctctattcgctgtattttttctttaatttataaacttttagtttaacatgc

SEQ ID NO: 72 - DNA, 2295 bp, Homo sapiens
agaccgcgagcgagagcgcccccgagcagcgcccgcgccctccgcgccttctccgccgggacctcgagcgaaagacgcccgcccgccgcccagccctcgcctccctgcccaccgggcccaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccagctccaccgctctgaaggggatctgcagagctcagtcagagggcagaccctgtgaatataactccagaatctaccaaaacggggaaagtttccagcccaactgtaaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgacgaggatagtatcaaggaccccatggaggaccaggacggcctccttggcaaggagctgggattcgatgcctccgaggtggagttgacgagaaacaatgaattgattgcagttggaaaaggcagctcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttgctcattcttgaggagcattaaggtatttcgaaactgccaagggtgctggtgcggatggacactaatgcagccacgattggagaatactttgcttcatagtattggagcacatgttactgcttcattttggagcttgtggagttgatgactttctgttttctgtttgtaaattatttgctaagcatattttctctaggcttttttccttttggggttctacagtcgtaaaagagataataagattagttggacagtttaaagcttttattcgtcctttgacaaaagtaaatgggagggcattccatcccttcctgaagggggacactccatgagtgtctgtgagaggcagctatctgcactctaaactgcaaacagaaatcaggtgttttaagactgaatgttttatttatcaaaatgtagcttttggggagggaggggaaatgtaatactggaataatttgtaaatgattttaattttatattcagtgaaaagattttatttatggaattaaccatttaataaagaaatatttacctaatatctgagtgtatgccattcggtatttttagaggtgctccaaagtcattaggaacaacctagctcacgtactcaattattcaaacaggacttattgggatacagcagtgaattaagctattaaaataagataatgattgcttttataccttcagtagagaaaagtctttgcatataaagtaatgtttaaaaaacatgtattgaacacgacattgtatgaagcacaataaagattctgaagctaaatttgtgatttaagaaaa

SEQ ID NO: 73 - DNA, 1069 bp, Homo sapiens
agcccgcgacctttatcccgcgcgttgcggtcaagatggcgctggtggcttctgtgcgagtcccggcgcgcgttctgctccgcgcgggggcccggctcccgggcgcggccctcgggcggacggagcgggcggccggcggcggagacggcgcgcggcgcttcgggagccagcgggtgctggtggagccggacgcgggcgcaggggtcgctgtgatgaaattcaagaaccccccagtgaacagcctgagcctggagtttctgacggagctggtcatcagcctggagaagctggagaatgacaagagcttccgcggtgtcattctgacctcggaccgcccgggtgtcttctcggccggcctggacctgacggagatgtgtgggaggagccccgcccactacgctgggtactggaaggccgttcaggagctgtggctgcggttgtaccagtccaacctggtgctggtctccgccatcaacggagcctgccccgctggaggctgcctggtggccctgacctgtgactaccgcatcctggcggacaaccccaggtactgcataggactcaatgagacccagctgggcatcatcgcccctttctggttgaaagacaccctggagaacaccatcgggcaccgggcggcggagcgtgccctgcagctggggctgctcttcccgccggcggaggccctgcaggtgggcatagtggaccaggtggtcccggaggagcaggtgcagagcactgcgctgtcagcgatagcccagtggatggccattccagaccatgctcgacagctgaccaaggccatgatgcgaaaggccacggccagccgcctggtcacgcagcgcgatgcggacgtgcagaacttcgtcagcttcatctccaaagactccatccagaagtccctgcagatgtacttagagaggctcaaagaagaaaaaggctaacgattgggctgccacaggcttacggccacacgtgcccctgtgggtcccagggaggtcttaaacaaggtatttttcaacttaaaagtactgccagcgtttcattttgcaaaaaaaaaaaaaaaaaa

SEQ ID NO: 74 - DNA, 3366 bp, Homo sapiens
cgtgggatttccagaccgcggctttctaatcggctcgggaggaagctctgcagctctcttgggaattaagctcaatctctggactctctctctttctctttctccccctccctctcctgcgaagaagctcaagacaaaaccaggaagccggcgaccctcacctcctcgggggctgggaggaaggaggaaaacgaaagtcgccgccgccgcgctgtcccccgagagctgcctttcctcgggcatccctggggctgccgcgggacctcgcagggcggatataaagaaccgcggccttgggaagaggcggagaccggcttttaaagaaagaagtcctgggtcctgcggtctggggcgaggcaagggcgcttttctgcccacgctccccgtggcccatcgatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaatgacaaaaaaaaaaaaaaaaaaaaaaaaaaaaaaaaaaaaaaaaaaaaaaaaaaaaaaaaaa

SEQ ID NO: 75 - DNA, 4491 bp, Homo sapiens
ccgaggcgagcgcgagcgaggtccagcaccatgtgctaggtcactcccagcgcgaggccacacctgggccgtcggagcagcccctcctcacttcaggggtcaccctccccagcacccattgccccaccatggctggggaccggctcccgaggaaggtgatggatgccaagaagctggccagcctgctgcggggcgggcctggggggccgctggtcatcgacagccgctccttcgtggagtacaacagctggcatgtgctcagctccgtcaacatctgctgctccaagctggtgaagcggcggctgcagcagggcaaggtgaccattgcggagctcatccagccggctgcacgcagccaggtggaggctacggagccacaggacgtggtggtctatgaccagagcacgcgggacgccagcgtgctggccgcagacagcttcctctccatcctgctgagcaagctggacggctgcttcgacagcgtggccatcctcactgggggcttcgccaccttctcctcctgcttccccggcctctgcgagggcaagcctgctgccctgctacccatgagcctctcccagccctgcctgcctgtgcccagcgtgggcctgacccgcatcctgcctcacctctacctgggctcgcagaaggacgtcctaaacaaggatctgatgacgcaaaatggaataagctacgtcctcaacgccagcaactcctgccccaagcctgacttcatctgcgagagccgcttcatgcgggtccccatcaacgacaactactgtgaaaaactgctgccctggctggacaagtccatcgagttcatcgataaagccaagctctccagctgccaagtcatcgtccactgtctggctggcatctcccgctctgccaccatcgccatcgcctacatcatgaagaccatgggcatgtcctccgacgacgcctacaggttcgtgaaggacaggcgcccgtccatctcgcccaacttcaacttcctgggccagctgctggagtacgagcgcagcctgaagctgctggccgccctgcagggcgacccgggcaccccctcagggacgccggagcctccgcccagtcctgccgccggggccccgctgccacggctgccaccacctacctcagagagcgctgccacagggaatgcggctgccagggagggcggcctgagcgcgggcggggagccccccgcgccccccacgcccccggcgaccagcgcactgcagcagggcctgcgcggcctgcacctctcctcggaccgcctgcaggacactaaccgcctcaagcgctccttctccctggacatcaagtctgcctacgcccctagcaggcggcccgacggccccgggccccccgaccccggcgaggccccgaagctctgcaagctggacagcccgtcgggggccgcgctgggcctgtcctcgcccagcccggacagcccggacgccgcgcctgaggcgcgcccacggccccgccggcggccccggccccccgccggctcccccgcgcgctcccccgcgcacagcctcggcctgaacttcggcgatgcggcccggcagactccgcggcacggcctctcggccctgtcggcgcccgggctgcccggccctggccagccggccggccccggggcctgggcaccgccgctcgactccccaggcacgccgtcgcccgacgggccctggtgcttcagccccgagggcgcacagggggcgggcggggtgctgtttgcgcccttcggccgggcgggcgccccgggaccaggcggcggcagcgacctgcggcggcgggaggcagcgagggctgagccccgggacgcgcggaccggctggcccgaggagccggccccggagacgcagttcaagcgccgcagctgccagatggagttcgaggagggcatggtggaggggcgcgcgcgcggcgaggagctggccgccctgggcaagcaggcgagcttctcgggcagcgtggaggtcatcgaggtgtcctgacccctccgctgccctcggccccgccgcccgcagccaggcccgttataaatgtatattatatataatgcaaagaaaggtaaatggttttactgggatttttatcgagaagtaaatatttcgattttttatttatttaagctgttcattctggcaatgatttggcaacagtgcgggtggtcctcgagctctatttttactgtctggtatttaaactgaaacatacgtttctaagcaatacgaggccaccttcagtcgcaagctgggtgccaggcctggggcccctcccagttcccccgccccaggaaacactgctgacctttgcaaaggctgccgagctttcgtgcactttttacataacaaaaaggtgaaaaaaaggaaaaaaaaacttctttgccacaaactgagccgcagaaccccccttctccccccacccacctcccctgctccctcccttctctgcgccggcctagggctctgcaccaaagccataggatggaggagcaggagctggtgtgccccggagaggtgcggccagccctccatcagctccaggcaccaaatcttggtggcaaggagggcaccccgctgcccgttgccccagagctgttctctggcaggggaggacaggcattgggcttcatggtgccagggtgttcagaggggctgagaaatagaacagtgtgtgtaggggcttcgggcagggggttctggaacgtcagatgaggtgcagcccaggggaggacagaggtgttagtgcccccaactcctgccagagccccagtccagccacagagtggctcagaaaggccattcctagagggctgcggccctcccttctcccttgcccatgcccccagagctgcctgccgggcagggtggcaccattgcaggagaggagcttggcctccgggggtcaggcaggaggcgcctggctagccagtgctggctccactgggcaggaagccctggacccccaggtatgaggagggggtggtcttagggttctgttccaggtctgccccgcccccctcccagccatgccccaggcagaacttggaattcaggtgtgcacctgcaggctgaggggctctgtgagcaggtgctgctcacacagggagttcaggcgccagccaagcccctgtgctgctgggataggcctgcttcacttagggagcactgcctcaagacaggtaaagccccctcgtttgcccccacccccatggggccgctcaggagagaaactcccattcacccctttcccagggtgctctctctctaggtggcatgccagcccccaaacacaagtggcttttgggcccaggtgggtcagcctgctgcccctgccccataccccctcgggccattgggacccctgcccttcagatgtcctagggtctaggagtggggccagtcactgtgggaagaggccaggggcttggccggagaggcagcccagggcaggacccagtcctgagtcctggagcagggccagggaggcgcccatcccgccccagccagccgccctctctgctgtttcttctatttgttcttcttttcacccacagctctgtgttcctgtcatccctcctttcagcaaaagtcctgttcccgttccctctgtccccacccactcctgttcccccaagaaaataagctatcgttgtatttgcaatctatggattagaggtttaagtatttattattattggttaattattattaattatgtaaatttgcctcccatatgtctgttgcgttgggtttctgaggagaccctgggtgaggaggatgcactggcttcccgcttctcgccccccacccctgtgctgtccgggagacagtggtctggggccactggttgggcccccttctcccttcccccttccccttgtcccttctgcaggccgttgaggggggctgtctgtctcagtctgtctctgctcccactcttgaggcactggttaccgcaaagtgagcagccagcaggggggcgaaggtcctgtgttggccactgcctcctccagtgctgcaggaggcgggctgaggccccacctggtggctttcacctgacccagccctgagtcctctccaagcctctctccggcccctcccacctggccactgcctcctccagtgctgcgggaggcgggccagggccccacctggtggctttcacctgacccagccctgagtcctctccaagcctctctccggcccctcccacctggccactgcctggcattgggatcgccccaaaatggacccggcccctcctgttatttgctgggaagtccagcggaggagagggtgcaggtcccccgctgagcctccagtctctgtagactgggctgccggcccttcagccccccttggagcccctcccgccacagccgcaccttctgctcccggcccctccctttgtatttggagacaatgtgttgtaataaagcttaaagtggatgttttcaaaaaaaaaaaaaaaaaa

SEQ ID NO: 76 - DNA, 2210 bp, Homo sapiens
gtttccggttgtcggaggcgaggcttgtcggctgtcaaaggggcggcccggcccggcccggaagctacagcagcggcgcggagactgcggggcgggccatggcggcgaacctgagccggaacgggccagcgctgcaagaggcctacgtgcgggtggtcaccgagaagtccccgaccgactgggctctctttacctatgaaggcaacagcaatgacatccgcgtggctggcacaggggagggtggcctggaggagatggtggaggagctcaacagcgggaaggtgatgtacgccttctgcagagtgaaggaccccaactctggactgcccaaatttgtcctcatcaactggacaggcgagggcgtgaacgatgtgcggaagggagcctgtgccagccacgtcagcaccatggccagcttcctgaagggggcccatgtgaccatcaacgcacgggccgaggaggatgtggagcctgagtgcatcatggagaaggtggccaaggcttcaggtgccaactacagctttcacaaggagagtggccgcttccaggacgtgggaccccaggccccagtgggctctgtgtaccagaagaccaatgccgtgtctgagattaaaagggttggtaaagacagcttctgggccaaagcagagaaggaggaggagaaccgtcggctggaggaaaagcggcgggccgaggaggcacagcggcagctggagcaggagcgccgggagcgtgagctgcgtgaggctgcacgccgggagcagcgctatcaggagcagggtggcgaggccagcccccagaggacgtgggagcagcagcaagaagtggtttcaaggaaccgaaatgagcaggagtctgccgtgcacccgagggagattttcaagcagaaggagagggccatgtccaccacctccatctccagtcctcagcctggcaagctgaggagccccttcctgcagaagcagctcacccaaccagagacccactttggcagagagccagctgctgccatctcaaggcccagggcagatctccctgctgaggagccggcgcccagcactcctccatgtctggtgcaggcagaagaggaggctgtgtatgaggaacctccagagcaggagaccttctacgagcagcccccactggtgcagcagcaaggtgctggctctgagcacattgaccaccacattcagggccaggggctcagtgggcaagggctctgtgcccgtgccctgtacgactaccaggcagccgacgacacagagatctcctttgaccccgagaacctcatcacgggcatcgaggtgatcgacgaaggctggtggcgtggctatgggccggatggccattttggcatgttccctgccaactacgtggagctcattgagtgaggctgagggcacatcttgcccttcccctctcagacatggcttccttattgctggaagaggaggcctgggagttgacattcagcactcttccaggaataggacccccagtgaggatgaggcctcagggctccctccggcttggcagactcagcctgtcaccccaaatgcagcaatggcctggtgattcccacacatccttcctgcatcccccgaccctcccagacagcttggctcttgcccctgacaggatactgagccaagccctgcctgtggccaagccctgagtggccactgccaagctgcggggaagggtcctgagcaggggcatctgggaggctctggctgccttctgcatttatttgccttttttctttttctcttgcttctaaggggtggtggccaccactgtttagaatgacccttgggaacagtgaacgtagagaattgtttttagcagagtttgtgaccaaagtcagagtggatcatggtggtttggcagcagggaatttgtcttgttggagcctgctctgtgctccccactccatttctctgtccctctgcctgggctatgggaagtggggatgcagatggccaagctcccaccctgggtattcaaaaacggcagacacaacatgttcctccacgcggctcactcgatgcctgcaggccccagtgtgtgcctcaactgattctgacttcaggaaaagtaacacagagtggccttggcctgttgtcttcccctattttctgtcccagctcatccgtgtctctgaagaacaaatatgcttttggaccacgaaaaaaaaaaaaaaaaaaaaa

SEQ ID NO: 77 - DNA, 9732 bp, Homo sapiens
gctggaggcaggaatgtgcctgcttactaatgagcgacgtgcaccggcgcgcacggcccggccaccgctgcggctgcggcggccggcggcggcccgttgtcaggtggagcctttgaattttttaaaagaccatagtaatcagatctacttgaaaaattaagtgaattgatttggttggaatgctcttttaccgatgctttaacatacagccactagattcccagaagttgtgcatgagttcctgtgaggagagttggcatcttggaaatcaaagacggttgtgatgcagttttttgatatgaggataacaggaagcgtcaggacaatcatgcccataagaactctaaactgttcagatcattttatcttagtgaaactggcaattgaactttgctctagttgatgggcaaaatttctcctagactattcatcatcccaatttcatcctagttgaaaattttcaaatgccataagaaatctttatagatttgcacttagcttttggatggacgtttctacaatggagagaactgtgttatagccctggtccaaggacattactagctaatgcccatcgactgtggtgtgcgtgtggaaggttccaaagagaaggagcaatcagcaagtttgcagacaccctggaacatggaagcaaccaagctttaagaagcacagctttggagacactccatgagtctgcactgctttcaggggaactagcacttaagaccttgtgtaacaaaatggacactggggacacagctctaggacaaaaagctacctcaaggtctggagaaactgataaagcatcaggtagatggagacaggaacaatcagctgttattaagatgagcacttttggcagtcatgaaggacagcggcaaccacaaatagagcctgagcaaatcggaaacacagcatcagcacaactgtttggttctgggaaactggcctcccctagtgaagtggtgcagcaagtcgcagagaagcaatatccaccgcatcgtccgagtccttactcatgccaacactcactctctttccctcagcactcattgccacagggggtcatgcacagcaccaagccacatcagagcctcgaaggtcctccgtggcttttccctggccctttgccatccgttgcctctgaggacttatttccttttcctatacatggccacagtggtggttatcctagaaaaaagatttcaagtctgaaccctgcttatagccaatactcccagaaaagtattgaacaggcagaagaggctcacaagaaagagcacaaacccaaaaagcctggcaagtacatttgcccttactgcagcagagcgtgtgccaaacctagtgtactgaaaaaacacatcaggtcccatactggggagcggccatatccatgtataccttgtggtttctctttcaagacaaagagcaatttgtacaagcacaggaagtcacatgcccatgcaattaaggcaggattagtacctttcacagagtcagctgtatctaaattggacctagaggctggttttattgatgtagaagcagaaatacattcagatggtgaacagagtacagacacagatgaggagagttctttatttgccgaggcttctgacaaaatgagtcctggtccacccatcccactggacattgccagcagaggcggctatcatgggtcattggaagaatcattgggaggtccaatgaaggtgccgattttgattatccctaaaagtgggattcctctccctaatgaaagctctcagtatattggccctgatatgctaccaaatccatctttaaatactaaggctgatgattcgcacacagtcaaacagaaacttgcactaagactgtcagagaaaaaaggacaagattctgagccatcgctcaaccttctgagcccgcacagtaaaggaagcactgattctggttacttttctcgctcagaaagtgctgagcagcaaataagccctcccaacacaaatgcaaagtcttatgaagaaatcatctttggaaaatactgtcggcttagtccgagaaatgcactcagtgttacaaccacaagtcaggagcgtgccgcaatgggtaggaagggcataatggaaccattacctcacgttaacaccaggttagatgtcaagatgtttgaagatcctgtttcacagctgatcccaagcaagggagatgtcgaccccagtcaaacgagcatgctgaaatccactaagttcaacagtgagtccagacaaccccagattattccatcatctatcaggaacgaaggaaaactttatccagcaaacttccaaggcagcaacccggttctcttagaagctcctgtagactcttcaccccttattagaagcaactcagtgccaacttcttcagcaactaatctaactattcctccttctttgagaggaagtcactcatttgatgaaaggatgactggttccgacgatgtattctatccagggaccgtgggcataccccctcagcgcatgctaagaagacaagcggcatttgagctgccttcggtacaggagggccacgtggaagtcgagcaccatggcaggatgttgaagggtatcagcagttcatccctgaaggaaaagaaattgtctcctggggacagggttgggtatgactatgatgtctgtcggaaaccctataagaagtgggaggactctgaaacaccaaagcaaaactacagggacatttcctgcttgagttctttaaagcatggtggagaatatttcatggatcccgtggtgccattgcagggagtaccaagcatgtttggaactacctgtgaaaacaggaaacgccggaaagagaagagcgtaggggatgaagaggacacgcccatgatctgcagcagcattgtaagcactcctgtgggcatcatggcttccgattatgaccccaaactgcagatgcaggaaggagtcaggagtggatttgccatggctggacacgaaaacctttctcatggtcacacggaacgctttgacccatgtcggccccaactgcagcctggaagtccatctcttgtgtcagaggagtcaccttcagccattgattcagacaagatgtcagacctagggggcaggaaacctcctggaaatgtgatttctgtgattcagcacaccaactcactgagccgacccaattcatttgaaaggtctgagtcagccgaacttgtggcttgcacacaggataaagccccttccccttcagagacttgtgacagtgagatttcagaagccccagtgagtcctgagtgggctccacctggggatggtgcagaaagtggggggaaaccctctccatctcagcaggtgcagcagcagtcctatcacacacagcccaggctagttcggcaacacaacatccaggttcctgagattcgagtgaccgaggagcctgataaacctgagaaggagaaggaagcccagagcaaagagccagagaagcctgtggaagaatttcagtggccccagagaagtgagaccctttcccagctccccgcggagaagttgccacccaaaaagaagcgtctgcgacttgcagatatggagcactcctcaggggagtccagctttgaatccacaggcacaggcctctcccgcagccccagccaagaaagcaacttgtcccacagctccagtttctccatgtcttttgaaagagaagaaaccagtaagctttctgcacttcctaagcaggatgagtttgggaagcattcagagtttctgactgtccctgctggttcatactcattgtctgtcccaggccatcaccaccagaaagagatgcgacgctgctcatcagagcagatgccttgtcctcacccagcggaagtcccagaagttcggagcaaatcatttgattatgggaatctgtcccatgctcctgtgtcgggagcagcagcctccacggtatcaccgtccagggagaggaagaaatgctttctggtgcggcaagcttccttcagtggctccccagaaatctcccagggcgaggttggcatggatcagagcgtgaagcaagagcagctggagcacctgcatgctggcctccggtccgggtggcaccatggcccgcctgctgtgctgcctcctcttcagcaagaggacccagggaagcaggtggcgggtccttgtcccccgctgagctcggggccactgcacctggcccagccacagatcatgcacatggacagtcaggaatctttgagaaatcccttgatccaaccaacatcctatatgacaagcaagcacttacctgaacagccacacttatttccacatcaagagacaattccattttctccaatccagaatgccttgtttcagtttcagtatcctacagtttgtatggttcatttaccagctcagcagcctccctggtggcaggcacatttcccacatccctttgctcagcaccctcagaagagctatggcaagccctcttttcagacagaaatccattcgagctatcccttagagcatgtggcagagcacactggaaagaaacctgctgagtatgcacacacgaaagagcagacctacccatgttattcaggagcatcagggctacacccaaagaaccttcttccaaagtttccatcagaccagagcagtaagtcaactgaaacgccctctgagcaggttcttcaagaagattttgcctcggcaaatgctgggtctttgcagtccctcccaggaacagtggttcctgttcggatccagacgcacgtaccatcctatggaagtgtcatgtacacaagcatttctcagatacttgggcagaatagccctgccattgtcatatgcaaagtcgatgagaatatgacccaaaggacactggtcaccaacgcagccatgcaagggataggattcaacattgcccaggtgctggggcagcatgcgggcttggagaagtaccccatttggaaagcacctcagactttgcccctcggcttagaatcctccatccccttgtgtttaccttccacctctgacagcgtggccaccctgggaggtagcaagcgaatgctttctccagccagtagcttggagctcttcatggaaaccaagcagcagaaaagggtcaaagaagaaaagatgtacggacagattgtggaggagcttagtgctgtggagctgaccaactcagacatcaaaaaggacctctcccgcccccagaaaccccagctggttcgacaaggatgtgcttctgagccaaaagatggcttgcagtcagggtcatcttccttctcctcgctgtcgccctcctcatctcaagactatccttctgttagcccgtcttccagggagccattcctgcccagcaaggagatgctttccggttcccgggcaccacttccggggcagaagtccagtgggccttctgaaagcaaagaatcttcagatgaattagatatcgatgagacggcatcggacatgagcatgagcccacagagttcttcattaccagcaggagatggtcagctggaagaggaagggaagggccacaagcggcctgttggcatgctggtccgcatggcctctgcccccagcgggaacgtggcagactcaactcttcttctcacggacatggcagatttccagcagattcttcagttccccagtctgcggacaacaactactgtgagttggtgcttcttgaattatacaaaacccaattatgtgcaacaggccaccttcaaatcctcggtttatgcttcatggtgcattagttcctgtaatccaaacccatcaggattgaacaccaagaccacgctggctcttctgaggtccaagcaaaaaatcactgcagaaatttatactctggctgctatgcataggcctggaaccggcaagcttacatcatcaagtgcttggaagcagtttactcagatgaaacctgatgcgtcctttttatttggcagcaaactagaaaggaaactagtgggaaatatcttaaaggaaagagggaaaggagatattcatggagataaagatattggatccaaacaaactgagccaatccgaattaaaatatttgaaggagggtacaaatcgaatgaagattatgtatatgtcagaggacgtggccggggaaagtacatttgtgaagaatgtgggattcgctgtaagaagccaagcatgctcaaaaaacacatccgtacccatactgatgttcggccttatgtatgcaagttatgtaactttgccttcaaaacgaaaggaaacctaacgaagcatatgaaatctaaagcacacatgaaaaaatgcctggaattgggagtctcaatgacatcggtggatgatacagaaactgaggaagcagaaaatttggaagatttgcacaaagcagcagagaagcatagcatgtccagcatttcaactgatcatcagttctccgatgctgaggaatcagatggtgaggatggagatgataatgatgatgatgatgaagatgaagatgactttgacgaccagggagatttaacaccaaaaacaagatcaagaagcaccagtcctcagcctcctagattctcctccttgcctgtgaatgttggcgccgtaccccacggggttccttcagatagttccctgggacattcttcgttgatcagctatttggttactttgccaagtattcgagttactcagcttatgacacccagtgattcatgtgaagatacccagatgacagaataccagaggctattccagagcaaaagtacggactcagaaccagacaaagacagattggacatacctagttgtatggatgaggagtgcatgctaccttcagagccaagctcctctcccagggacttctcaccctcaagccaccattcctctccaggatatgattcttcaccctgtcgagataattcaccaaagaggtatctgatacccaaaggagatttatctcccaggagacatttatcacctaggagagatctgtcacccatgagacatctttcaccaagaaaggaagctgcattgagaagagagatgtcccaaagagatgtttcaccaagaaggcatttgtctccaaggaggccagtgtctcctgggaaagatatcacagcaagaagagacctctctcctagaagagagagaagatacatgaccacaataagagcgccatctcccagaagggctttataccataacccaccattgtccatgggacagtatttgcaagcagagccaattgtattggggcctcctaatttaagaagaggattacctcaggttccttacttcagtctctatggagaccaagaaggtgcttatgaacatccaggctccagccttttccctgagggtcctaatgactatgtcttcagtcatcttccactccactctcagcaacaagtgcgagcccctatccccatggtgcccgttggtgggatccagatggttcactccatgccgccagccctttccagtttacatccttcacccacattgcccctgccaatggagggctttgaggagaagaaaggcgcgtcaggggagtccttctccaaggacccctatgtgctttctaagcagcatgagaagcgaggtcctcacgctttgcagtcatctggtccacctagcactccctcctctcctcggctgttgatgaaacagagcacttcggaagacagcctaaacgcaacagagcgggaacaggaggaaaatatacagacttgtacaaaagccattgcctctctccggattgccacggaagaggcagctctgctcgggccagatcagccagcgcgggtgcaggagccccaccagaaccccctgggaagtgcacatgttagcattagacactttagtagacctgagccaggtcagccctgtacctcagccacccaccctgacttgcatgatggtgaaaaggacaattttggtacatcacagactccattagctcactccacgttttacagcaagagttgtgtggatgacaagcagttggactttcacagcagcaaggaattatcttcaagcacagaggaaagcaaagatccttcatcagaaaagagtcagctacattgatctatgatgcatggagactttcatttccacattttcccatttttttgtttttgtttttctagaaatggaggtaatccagtttatagcatgcctgtcctaagttacagtagtttgctattatatatacttttgttatatcaaaagaattaggtaaattaacaagtcatcatgagcctgaccaaaacaaaatttgaaattaacctattgggtctggtacttttaaaattgtacagatgtttgtgccttttctttactttgcttatattcttataagcattttttagcagtaatttgtacatattttagaatttgtgtatctgctttgtaataaatgtaatttctttccttttttggacacttggatctaaatgatgtaaagcaaaacagcatcaatatatatgtgaggttgcactaaaacatatttttatatgattaaaactgaacagcttttatgtacagctctgattctgtaatactaatatttatttactttgtttcataaattgtacattttttcttaatgttgtggattgcttttctatgtgaagcatgggatttactgttgcgtaactagaacaaaaatgtacattgtaaacaagatatttaaactagagtatcttattctgcacttatgcattagttaaaaaaagataaaggatgtatcagtcagttcttaactcttgtatatttttttgtctcttgtttgctggattgactataacttaagtgctgattgtgattttaaaatgatagtaccgtaaagcattaaagtaaacaatgtgctattgtgagttttttcaaagctttataaatcagttataaataatattaaaagtatttggtcttatgtgaacatgttgatctatatactcatctaaaaatatgggaaaacattccaccccatgtaaatatgtacaagtgcatcactggtacaattttatgtaactcagttggacactaggttgccacagacctatgctaggtgtctttaaaaaattaaggtgacaaagcacatgggactgtgtagagcttggttatcggccggcccggtggcttggcaggcagtgctgtgcgctgctcatggagaagacctgggcttagcaatctccttagttcttgctacacaggatggtgactggaactaaggctacacagagggtcgcacttggactctgagggttgggtgtggaagggggaaaaggagatggagacctgctccccagctcttcctgtcagccggtttacatgggaacagggttaacatctgtgttaggggaggtcaccttaccctttttcataggggaagagtgtcacactcctggctatctcagggggaatggggaaaagaatctttcaagggcaaagaactcgtgggaggatgtctgttgtatgtaatactcacaatggcttttggttagtgttgaaggtgggaagagcatttgtaggtccagaagagtgaaagagagggaggggtgcagcaacatgtgcacaggcacgcacatgtgtgcacgcacacatacaatctgggttatctttgtgctatatagtggattataattctgtgaaaccaagtttgtatattgaattacattaaggagtgttctttaaaaagagaaataaatatacaattacatgcttgaaaaaaaaa

SEQ ID NO: 78 - DNA, 15245 bp, Homo sapiens
cgctccgcccgcccggccgccccgagccccgagccccgagccccccgcgccgggcccgggcggcagcggcggcggcggcggcggcggcgcgcccggccccctcccccggcgccggccacgggaggcggtgatgcgggcgcgggcggcctcggctgcgccgagagcggagacacaggctcaagatggcagattccgactgaggctgggggggccgagctcgcgcgccgctttcccgtccccgttgccatgaaccgcggacaccccggccccgatggcccccgtgtacgaaggtatggcctcacatgtgcaagttttctcccctcacacccttcaatcaagtgccttctgtagtgtgaagaaactgaaaatagagccgagttccaactgggacatgactgggtacggctcccacagcaaagtgtatagccagagcaagaacatccccctgtcgcagccagccaccacaaccgtcagcacctccttgccggtcccaaacccaagcctaccttacgagcagaccatcgtcttcccaggaagcaccgggcacatcgtggtcacctcagcaagcagcacttctgtcaccgggcaagtcctcggcggaccacacaacctaatgcgtcgaagcactgtgagcctccttgatacctaccaaaaatgtggactcaagcgtaagagcgaggagatcgagaacacaagcagcgtgcagatcatcgaggagcatccacccatgattcagaataatgcaagcggggccactgtcgccactgccaccacgtctactgccacctccaaaaacagcggctccaacagcgagggcgactatcagctggtgcagcatgaggtgctgtgctccatgaccaacacctacgaggtcttagagttcttgggccgagggacgtttgggcaagtggtcaagtgctggaaacggggcaccaatgagatcgtagccatcaagatcctgaagaaccacccatcctatgcccgacaaggtcagattgaagtgagcatcctggcccggttgagcacggagagtgccgatgactataacttcgtccgggcctacgaatgcttccagcacaagaaccacacgtgcttggtcttcgagatgttggagcagaacctctatgactttctgaagcaaaacaagtttagccccttgcccctcaaatacattcgcccagttctccagcaggtagccacagccctgatgaaactcaaaagcctaggtcttatccacgctgacctcaaaccagaaaacatcatgctggtggatccatctagacaaccatacagagtcaaggtcatcgactttggttcagccagccacgtctccaaggctgtgtgctccacctacttgcagtccagatattacagggcccctgagatcatccttggtttaccattttgtgaggcaattgacatgtggtccctgggctgtgttattgcagaattgttcctgggttggccgttatatccaggagcttcggagtatgatcagattcggtatatttcacaaacacagggtttgcctgctgaatatttattaagcgccgggacaaagacaactaggtttttcaaccgtgacacggactcaccatatcctttgtggagactgaagacaccagatgaccatgaagcagagacagggattaagtcaaaagaagcaagaaagtacattttcaactgtttagatgatatggcccaggtgaacatgacgacagatttggaagggagcgacatgttggtagaaaaggctgaccggcgggagttcattgacctgttgaagaagatgctgaccattgatgctgacaagagaatcactccaatcgaaaccctgaaccatccctttgtcaccatgacacacttactcgattttccccacagcacacacgtcaaatcatgtttccagaacatggagatctgcaagcgtcgggtgaatatgtatgacacggtgaaccagagcaaaacccctttcatcacgcacgtggcccccagcacgtccaccaacctgaccatgacctttaacaaccagctgaccactgtccacaaccaggctccctcctctaccagtgccactatttccttagccaatcccgaagtctccatactaaactacccatctacactctaccagccctcagcggcatccatggctgcagtggcccagcggagcatgcccctgcagacaggaacagcccagatttgtgcccggcctgacccgttccagcaagctctcatcgtgtgtccccccggcttccaaggcttgcaggcctctccctctaagcacgctggctactcggtgcgaatggaaaatgcagttcccatcgtcactcaagccccaggagctcagcctcttcagatccaaccaggtctgcttgcccagcaggcttggccaagtgggacccagcagatcctgcttcccccagcatggcagcaactgactggagtggccacccacacatcagtgcagcatgccaccgtgattcccgagaccatggcaggcacccagcagctggcggactggagaaatacgcatgctcacggaagccattataatcccatcatgcagcagcctgcactattgaccggtcatgtgacccttccagcagcacagcccttaaatgtgggtgtggcccacgtgatgcggcagcagccaaccagcaccacctcctcccggaagagtaagcagcaccagtcatctgtgagaaatgtctccacctgtgaggtgtcctcctctcaggccatcagctccccacagcgatccaagcgtgtcaaggagaacacacctccccgctgtgccatggtgcacagtagcccggcctgcagcacctcggtcacctgtgggtggggcgacgtggcctccagcaccacccgggaacggcagcggcagacaattgtcattcccgacactcccagccccacggtcagcgtcatcaccatcagcagtgacacggacgaggaggaggaacagaaacacgcccccaccagcactgtctccaagcaaagaaaaaacgtcatcagctgtgtcacagtccacgactccccctactccgactcctccagcaacaccagcccctactccgtgcagcagcgtgctgggcacaacaatgccaatgcctttgacaccaaggggagcctggagaatcactgcacggggaacccccgaaccatcatcgtgccacccctgaaaacccaggccagcgaagtattggtggagtgtgatagcctggtgccagtcaacaccagtcaccactcgtcctcctacaagtccaagtcctccagcaacgtgacctccaccagcggtcactcttcagggagctcatctggagccatcacctaccggcagcagcggccgggcccccacttccagcagcagcagccactcaatctcagccaggctcagcagcacatcaccacggaccgcactgggagccaccgaaggcagcaggcctacatcactcccaccatggcccaggctccgtactccttcccgcacaacagccccagccacggcactgtgcacccgcatctggctgcagccgctgccgctgcccacctccccacccagccccacctctacacctacactgcgccggcggccctgggctccaccggcaccgtggcccacctggtggcctcgcaaggctctgcgcgccacaccgtgcagcacactgcctacccagccagcatcgtccaccaggtccccgtgagcatgggcccccgggtcctgccctcgcccaccatccacccgagtcagtatccagcccaatttgcccaccagacctacatcagcgcctcgccagcctccaccgtctacactggatacccactgagccccgccaaggtcaaccagtacccttacatataaacactggaggggagggagggagggagggagggagagaatggcccgagggaggagggagagaaggagggaggcgctcctgggaccgtgggcgctggccttttatactgaagatgccgcacacaaacaatgcaaacggggcaggggcgggggggggggggggggcagagggcagggggacgggtcgggacaccagtgaaacttgaaccgggaagtgggaggacgtagagcagagaagagaacatttttaaaaggaagggattaaagagggtgggaaatctatggtttttattttaaaaaagaaaaaggaaaaaaaaaaagtcaataacaaaaaacccagctcaagaacccattctacgccaaactggaaaggagaagagagcaacaggaagattccagaaacggggggccccagtttttgaagaactttatgaacttttcaaagattattttcatatggcagcaagtgatacggaagactgctgtcagggacacctgatatggaaatcaaatagatttttaattaattgaacataagatttagggatttttccagaactcgaaagggtcaacagccctccagaatgtcgggctgcagcctgaggaggctgatgtttggagctggtgtgggattggcgaagcccagtccgggctccctagtcaggaaagacgggggacggccaggctgctggaaggcccccgggggcgcggggcgagttttctttttctgagcactctggataaatccctaagcaacgttgtttctcaaatgtcattaataatgtgtgttgcaaactttaggtttttttcttttctgaaaatgtattttctctttgaatccacccctagtcgcgtagcgtagggctagcggtcgtcacagacaccctagtagaatgtagcactcagcacccttgtctcctaccttgtgttcaactccaatgataccaatagaatattcctcaatgtaattgcacaaaaaaaagcgatataacataggcatgtaaccaatgtggcggtgcaggtgtgcgggtgagcgagcacgtgtgggtgtgcacgcgccgccctccccgcgtggccctcggcgccgccaccctagctggcgcagtcttgacactgcatccttccctcctagtgccttaccgagcgacagacgcggcgtgagggtttacttccactggtactccaagaaactgaggctagtcagacacaatctcagctcttctgttgtgctgttgtaacagtttacgctggccttttttttttctttttctttttctttttaaaatgttaatgcccgttgtctttcctgggctgtttgctagcggaaggatgccagggaagccagcaggagctaggagagagtccgtggatctcgaaagaaatatgggagacagatgcccggcgggtgcgtctggagatggggacggcgggagttgagttgtggcagtagttgagttgtaatttgtgggcggaggcccagagagactccccacccttcacccctgccccactctgtccccagttccgccatttgtgaggccagaggtttccggactgttggcctcgccaggcagccgtctcccgccccaggcggcatcccccagtccctcccgcctccacgagagcctggagctctcagcctcgcccggggctccactctctcctccggctccctgggctgttttgctctaacgatcttgccagatccctccctctgtagacaaccaccaacctctgtttgctgttgaattctctcctcacattacccaggtctgctcaagacatgattttggttttggtttctgagggttctagtgggcagaaggttggagggacacttatgagggtggccgggggtctgacgctgcactttggaaaaactcacacagttgaatttccaaagaaatctgccctttgccctctttgcacctttgatacattctggaagttttctcaggctttggacacttctggggatggaggtgtggagaagtggggagttccctctcttcatagtaaataactctgaaatatgtgaatgtgaatggcaggagaatctggccaaggatggggccgaaaagggtggttctaattgtttgcttctgatgttgagtctttagctgaccccacaggcaggtttccaaggtgcaaagagatctttcccgagtcagcggccccatcctcatcctccctccctttacttcctcactgtgcagtctccctcaaggatctactgtgaaaggtgtgtttgtagtgatatccaacctaactcagtaacgaagtcgttacttagctcttagctgtgaaataactctggaaacttccccaccccaaccataaattcttacttataaagaaacaggtccccaaactggaaacagcttagtccaggcctcagcgagaaggaaggacaccatgactgctccatgctgggcacagccgggcagtcttgccaagtgcctgctggaggctgtgccggcaagaggcctgcagcaaggagattcccttccctcgggccattatcaatactgtctttatctggaggtggggaagcgcagccctctgagacagcaggacaatggtcagttcagagagggtgagggcagcaaacgcttcagaggacacagaagccagaggacccccccccgccccacagctgggtcagcctggaaaatccatctattagggactttttggcagccagatggcagcaatagcccattaggtctcatcccgagttccaagtcttggctgcaaatgagcctcagttcgccttactggagagcacccccagattcctgggcacagttcatttccagccctttctagatctgatcttttagggggaaagacagcttaaaatgttcttttcattttaaagaaaattattctgtctgcttaagttggaggctacttactctttcacctgacattttctttccttttattcttccagatcaggaatgaaatttccatgctgctcataaagataatattattgtactaattatttttattaccattgtaattatgatcattatgttgatattttagtcagggttttaaatgcacatttattccaagtatctttgtgttttctctttaatatttaaacttattctctctgtgagtatataagtagactggagggacatccagatgtccagttttgtcaggcaaaaaaaaaaaggaaagacttaggaagtaggaaaattgtttctgtcatctctatcccaacaagagacgtcaagaaagatccaccacagaacaaaagtttaaagaagaatcaaagccttgattgggcttctgacaacatggtcaccatcaaggttgtcattttctagatcccagaggcctgggatgcgacgtcaggtggcatctcatgggctcggggaatgtcgagtcactgactgtccagcccttagccagcttctctcccacatcctcagagctctcctgtgcttctgaaatctgttaactaaatctttggcttgcctctggtatttaagcaagaaaattccctcccagaggtgaccccatccgcttccccacaatccatccttttgccatcggcgcacctggggcgtggcttaggttcttcaatgcagggacatttgccccctcccagaagctgctgggcacagtgaggtggcgtaagagtgactggcaggtggtaccttccccaggaaatttcaccacaccacccagttcctcagcctgccccctccccctgtgatgcatgcccccagcacccaattctagccagctggaagtgggtggagggacagcaggaggccagagaaaccctgaacaaagctgggcggctgctcaggcatcacaggctgcaccccctctgaaagcatccccactgggctccggccacatcttcagtgcactgtgctgtgtgcgctgggtgctcacacgctgtccccagacccacaaagtgctaggccccagttgaagaaaggggtgaaatagccagcttcaccgaagggaagggaagggaagtattgggcgatgccagccccacagacgctcagcaaacattagtgcacattctcctagtcctcacccaatggcctcctctacccccatgcatggagctgccacatcagaagccccaagagaagctccctgcaggagaggccagctccctggatgcccaattgcatacctggccgaatctgccattgagtcaccttagcaaataggctgctgtcactaggaccaagctctcaagcagagggatgccaacctagtccttacttagcccacgaatcatctagagcatcctctagtcttttgtgggctccctccttcccatttgaagagacattgttcagaggaagaggggaagatttgaaatgtcaggtcacggaggagtgtttaactggagcctggtgaaccgcagggcaatttgcttctgctcactgggttctgactggcccgtctggacgtgggcccccatgtctctgtgcttagggcctcttcatgatgttttggatgtttccaagggaagtgggtgagcagatcaaggggtgggagagtcgaggcttgatgccagttaatactgtgaagtggagcgtgcggtcagtggaattcagaggaaaaagaagggttggagcaaagcggcattcatctcctggactgttagcctttctagtcttcctggtggctgaggtgttcacgggctgggggagccagctgacctttgtcctcttcaacctagaagactcagcccgcccagacaccaacgtgtgagacggatggacatcaggaagggaaggggagattagcccaactgctgacagaacgatttcccttggttggaccttgggaatggcaaacactcatattggaacaagcttggggtggaagatttaggccgtgtgagcatgtgtgagtgagtggaacaaactttcttggaaactggagggaggagatgaggaggcttcgggaagtattactgatggctcatggttgagagagcgacgtggggacccagctcgccccagcttttgtcccaggttctctttgtctgatgctgagggcagggtggggtgtgggaccaccactcttgttggcctgtcaagtagaccctaggacagaaaatggaaagaaggaaatggctcggtgctctcaactagcagagagaattgaggagaggtaagggttccttctgcaggccagcctgggactccacagcgccagcaggagtgacttggccacaagacattccagccccagggactttgcaggcttcattccctgtctgtgtcttttccttctggtgtgttttacagacttctgatggggaagcttcaaacttgagcaggccagagatgtccttaccaaattggaaaggaaggtgaaactgttcctttctttagccaaagaacccttctcaaagacgcctccagaaatggacaaaatggccttcccttcgttcctttccaggcaataatgacatcattagtgatgcaattctatttgtctttctctttcctctctgtccttttttttaaaaaaaaaaaatgcatttatttcaaaactgtgctattcttttaagaggagtggaggtgaccccttcgatgctgctgctatcgggagacaaggtgccataccaatacgtgggcttgactaatcccaggccaccatgggagagagcaaagcagggctgccaggagttcagttgcatcaagggcgtagagcacgcgggggctgggctcggaatagcagtacttttccactttgatgccttagaactctcacttctcatctccacagaccagactcagtaaaatctcaggccactagagaatggaaggcggtgaaacaggatttaaatgcaaaaaaaacctattggaggcttttggcaccgtggctcactagagggacccagcatagtagaggtttctcttgttgcagcttctgaaaagttcaaaaaagaactccaggccgttcttccctcaaacccagtgagagtttgcagagaagtgccccctgcagggctcccgtcccagaacaccagcaccagagagggtcttcccgatgccccccgctggacttgcccaagcctctgggagcccctcatctcagatccctgtgttgaacatgacactgactgtcccttatttgttaaaatttgcaatatctctcaagtaaataatagccaacatttgttgaatgctttcatgactcccgggctaaggcctttatgagcgttgtctcaaggggccccaacagccatcccacagggagggggataacagcccccatttatagataagggagctgaccggatgctctgagaagtggcaggtgttgaaggaaggataaagcagtgatgggccagaatccccaaggttcccttttttgttcatcaggcccttcctgagatgtgatttttaatcttttaactttttttaattaatagcaatgcgtggcctcatatttctatgaaccatttagtgatactcccgcttcctgcatgccacacactgtgctgggaattgctcatgggttgtcctgtttcatcttctccgtagccctgtgagatcggcaatattagtccccctacagccaaggaaactgcccagagccacacaactcttgaggggcgaggagggcttgaacctgagtctgcccagctccagaactgagcttgcagccattagccacagctgtctcctgcatgtctgagcaaagaaaggcctttacacagcatcaccctgtgccatcccatgcaccgtgggactcagctaaaggactgtgcaaagagggggctcctgagttggatttaggcaaaaggggcagaattcgtttgatttttagagaaaatctctggagagtttcttttgattcatagaattccttttagatttctttccagcataccaactagctttagtagtgctgctacaaccagctcttataagtaagagtgaaaaagtattcttttcttctttaaaaaataagtttttcttgcttatagttaattctagaaaggcaatactaaaggtatatatttttttcaaaatgctattttttactgcacttgataattatcctgacagctctgatctctgtaatagattcactcttcagctctgggcagaaccagaggcagggttcacaccaaatttgtaaataccatatgtgggtctggtgtccaggaacttttttctttctgttaaaaaaaagaaaaaaaaaagaaaaaaaaaaagaagtagaggtggaagaaagacaagacttagaggaacaaaagaatgttttcttttgagatactcttctcaaagaaatagcaacaattgtataaacaggaaaaccagccagctttcatgataaaaggaaggcgtgtctcttgccctggtatgagattaacagaaatacagatgcatttttattttgattgaaagatggtgagaatgtagaaatgcttaggactagatttttaattttttaaaataactattatcatttattatgaaatatttgttcagttgttttgagtgggtttcttgttccttttttcatttaaaaccttctttgttgactggctccaggcttgtttgcctaaattcttaggtagtttacacaagttctagaatcttttagaactttaactccattggaagcaaacctaactaatcggagtttgagatcctggttggtttcaataggtattctggaattctggcagaacacctaaagattttttttttttttagaaggttttagatacattatcttacacaaactgtgacctaatggcaataattacctcaaatgtgggcattcatcctggttttagccttttttgaaatcatgtagccagcttgatcttggaatttaaagactatgaattctctgtgggctgaaaataatgattacttcatacccccggtcatcgttgcttaagtgaattctgaaaatagctcatctttacaacaaaaattaaaccaaggaagagattattctttgtgtgttgtactcaaatgcgatgttcaaatgcacatgttaagtatatatgtttttagctactgtaaaatgctgttagccttctaagctatcaaaacagtcacattttaaatgagtaaactaaacaattgactgtggatacttaagcatatttctggctacgttttatagttaaagtgttttatagtttacatttagactggtactttttaaagaaaagttctgtttataactgacatccgcaaaccccagtgaatgcctcttagttggaggttgtgtctcccccaaggcaagtgtgttgtcccagactcttctgtagtccagcatgcgcacttccctctggattattactttccacgtgaactcaagagaacatgaaaggcaatccagatggagggaaaaggtgtgagtccgcagcccgggccagatgcgaaggtctcatgcgtgtcgtctaaacacttgtcttcaaggccttctctctgacatcttggaagagtcattgagaacagataacctggttcattgatttttgtcttgatttgaatatttaacttattaatagatccactgatttccaggcaccaggcagtagaagagactgggattcaggtgaccatgaaggcacagctgctacttctgggccgggggtgatattttgatcagcgttttgtaggggaggaccatatacccctattcccatggtcgctggctgggttttccatatatctgctgtcatttattcgttttccccttaaaagcaaaatcaatgtaaaaggctatgtttacgttttactcattgtccagcttagactcaaagtctagttcggtgggagggggaccttagcatcctctcagagatggtcagggctgagcaggaggaggcagagacagaggggcagctcagcctggtccattgagacccactctaaacagacatcatatttggaacaagaagatgcttcgagacaggaatgggccccactgtcatgcagaaacagactgggggaatggcagtttccctgagtcttggtttcttttatgttttctcttgtgccaccaccaaactgcagaggacctgctgtgacctaaagggcattcctttagcagataagaccttgaaaactgcaaaacacctgggaccagggagcttttaaaaaatacaaaaaaataccacatttgctttttccctgtgaactgtattgacagcgtgttcttaggacagtcttttggtggaaatgttactgtaaaatagttttcatctcacccctcctaatcatactcccactttcctgtttgtgtggtggtgttgctgttttttcctttacatgaattaaccaaatgaattttgtgtcattgtttttggggcttatatttttaaaacatagaaattgccttttgttcatttgaaaagtaagtatgttgtatctgaaaaagggctctgcctctgctctccctcgcttccttgtaaccaatctccaaacgaatctctcctggcaccgcccccttccttatatagggtcactgtccccggggccacctctgcctccaccctgctgtcaccactgccctgggccaaggcacccaggactcccagaaagcgcgagagccagcaagaaggccccactcagccttgagactggtggtcacacctccctgtcagagtcgcctgctgggctgaaggggcaatggattgtcattgttgaaattgtttggctcaggttataaggaggaacttgggaagtagaaagtgacttgaccatgtgcatccttggtagcttcctgtaactaacaaatggaacagagagcacacccccgccccgccccaccccaagcagatgttcccgtcagcgctgccctgagtcagtcggtcccccgtttctgctctcctcccttttgtgttcctgctcacttcaagcttcttccatggactttccagggcacagtcatctctagcccccaaatcatctcttcatcctctgtgtgtgcatttttttaaccaaatgaaatagacaagaaagtcatactttggggcagcagaatttctaatttagtagaatcactgtatagagatagatgttgatatatatgtttgtgtatatatatcaaaccaaattggataggagaagtatagctttacagatgaggagaaggagctctttagaggtcggagtcaagactggtgtcttggacgtgcatgggctgtgtcccaggccactccgcacacatggggctgaggcgtggcgccgggcccttgtcatccacctcaccacggcagagccagcaggccctgtagggtgctgctgctgtctcactgggtcccagcttcaagcgcatcagtgggtgacgggggcaacaaatcagagtgactggaagtttccatcccgttttgctttgaccacgtgtactgagctgcagcctctgatactctgtcacgtttccaaaaatggtatccattaggatagaaagagaatggatctgcagaaatgtttacctttcaactgctcatgaattcaggacactggatagaaagactcactccccaaaatgagaacagggaagaggagacccggcgacactaagtcaccaggtccaaggaacgtggctccctccccagggtcatctcacctagatctttctctcccaggtcatctcagctcaatctcaataaccctatgaaagccctggtctgttgtgttccttcaccgtacggtttctgtaataaaaagtgttaatccatgttaatctgtgtgaaaattattgcgtgcaacagtattttctcgtgtacctctttttcctatgtgaattgtccctcttttttatttataaatgtctacttttgtttttttaaagacaaaccaatgtgttgtagacctatatgtaacctattccttagtctcatattataggtatgttataagaatggatattttacttggctttagaatgttttacaagaaaactaattcttaactgatcaagtccttgctactaaaatgcttgtgtttttcatcatgacgtcgtgtgcttctaaattaatcattttcgttgtagaaaaatggagtgaatttatattagtcttggaaactaataatagcattgtaaatttatgagatgattttaacagaaaaaatatagaagaatatagttattttaattgtaatattactaactgtagggtgagaaaaaggggggggggtcccattgtggtgaactatgttatagcttgttactcatagtttctttttgatcattttttcggtctccgaggtgaaatgacttattaattaaaatttgtaaactcacatatgcatattgtatatgtgtagaaatgtaatcacactttgtcttggaattacattaaactgtttgaaatcactgtaaaaaaaaaaaaaaaaaaaaa

SEQ ID NO: 79 - DNA, 7385 bp, Homo sapiens
ggcgccgccggaccgcgccccaccccgcgcgcccgagcagggcgactgtcattagcttcctggacgggacccggggcgggatcctggtgtcctgaaaggggcccgggcgaccctaagaggaagaacttttgggggcggggtccccggtcccgcgtcccctgggcagccgctattgtctacgcgcctcgctgggcggcgcggggggcgtgatcgcggcggccccgggctctgggtgcggagacccaggcggggctgggcccagggcggcggcgggagaagccggggaagccgaagagcctggggaggaggagctgcgagcgcgggagacgagcaggagccgcgcgggccgcggcgagcgcgatgccggcggcggcgggggacgggctcctgggggagccggcggcgcctgggggcggcggcggcgcggaggacgcggccaggcccgcggcggcctgcgagggaagtttcctgcctgcctgggtgagcggcgtgccccgcgagcggctccgcgacttccagcaccacaagcgcgtgggcaactacctcatcggcagcaggaagctgggcgagggctcctttgccaaggtgcgcgaggggctgcacgtgctgaccggggagaaggtggccataaaagtcattgataagaagagagccaaaaaggacacctatgtcaccaaaaacctgcggcgagagggtcagatccagcagatgatccgccaccccaatatcactcagctccttgatattttagaaacggaaaacagctactacctggtcatggagctgtgccctgggggcaacctgatgcacaagatctatgagaagaagcggctggaggagtccgaagcccgcagatacatccgacagctcatctctgccgtagagcacctgcaccgggccggggtggtccacagagacttgaagatagagaatttgctactagatgaagacaataatatcaagctgattgactttggtttgagcaactgcgcagggatcctgggttactcggatccgttcagcacacagtgtggcagccctgcctacgctgcacctgaactgctcgccaggaagaaatacggccccaaaatcgatgtctggtccataggtgtgaacatgtatgccatgttgaccgggacgctgcctttcacggtggagcctttcagcctgagggctttgtaccagaagatggtagacaaagaaatgaaccccctccccactcagctctccacaggtgccatcagtttcctgcgctctctcctggaaccggatcctgtgaagaggccaaatattcagcaggcactggcgaatcgctggcttaatgagaattacacgggcaaagtgccctgtaatgtcacctatcccaacaggatttctctggaagatctgagcccgagcgtcgtgctgcacatgaccgagaagctgggttacaagaacagcgacgtgatcaacactgtgctctccaaccgcgcctgccacatcctggccatctacttcctcttaaacaagaaactggagcgctatttgtcagggaaatctgacatccaggacagcctctgctacaagacccggctctaccagatagaaaagtacagggcccccaaggagtcctatgaggcctctctggacacctggacacgagatcttgaattccatgccgtgcaggataaaaagcccaaagaacaagaaaaaagaggggattttcttcatcgaccattctccaagaagttggacaagaacctgccctcgcacaaacagccctcaggctcgcttatgacacagattcagaacaccaaagccctcctgaaggaccggaaggcctccaagtccagcttccccgacaaagattcctttggctgccgcaatattttccgcaaaacctcagattccaattgtgtggcttcttcttccatggagttcatccccgtgccaccgcccaggaccccgaggattgtgaagaaaccggagccccatcagccagggcccggaagcactggcatcccccacaaggaagaccccctgatgctggacatggtgcgctccttcgagtctgtggatcgcgacgaccacgtagaagtgctgtctccctctcatcactacaggattctgaactccccggtcagcttggctcgcagaaattccagcgagaggacgctgtccccgggtctgccatccggaagcatgtcgcctctccatactcctttgcatccaactctggtctcttttgctcacgaagataagaacagccccccaaaagaggagggcctgtgttgcccacctccggttcccagcaatggccccatgcagcctctggggagccccaattgtgtgaaaagccgaggccggttccctatgatgggcatcggacagatgttaaggaagcgccatcagagtctgcagccatctgcagataggcccctggaggccagcctgcccccactgcagcccctagcccctgtgaaccttgcctttgacatggccgatggggtcaagacccagtgctaacttgggccagcggggtttggggtatctctagaaaacagcaactgaacagagctccacacatctgtcagggtgtgagcactccaaggcctcgcgtggagcatccttagtcccacctgtagctgaatccacagacccaaagcctgcacaacccaacctcgcttagggacccccagagatgctggaatcgctaggagggttggctccaggggcagccaattcctatcattcagatcttccttcctcccaagtactcaccaaccccttccacttcccacttcccccaggcttggggggaaaacagggcatgagccttctggggcactcagattatggactgttaccagatctttcttcacgctgtgctacatgtgtgcctctcacagcagttggccacagttacagggagagaacaatatcacagtcattcatccaggccacgtttcctctgcggagtgtagcagccctgcctttcatagcagggattacctgaaggccagcaggagccgggggcaggcccaggatcctcagaggaagatggagaggagcttcggaccaagatcaaaccaaacagtggggaccccaacagaagagaaagactgaaggagacactcatttcccaagcaagattttgatagatttttgttgttgttgttgttgaaacatgctaatgattgaattatcttttccaaagattttttttaaatgtgatgtcggtaaattgaaataacataattttttaaaacttggatggagagatgagaagcaattccaccaaactcatgttttcacaggagggttcagtgtggagagcaaaaaagctgactgtggtgatttgctgagtgctgtggcccacaggcagggcaagtctcggtggccctgtgttcatcctgttgtttaaggcatagccctgatccttctggaaggcatagaacacggttacagctggttctgtagaaagagggaaaagatgattgtgacaattcagagcataactcagatggcgagaaggcagcattatctctgtgcgatgctgatttcaagctgcccacagaactggtgcggctcagagctcggcgagtttgctgggagctgggagcaggcttgcctggcagagaacctgttcagatacaggccagtttcttcttcgaggaaagccaagctcctcaaacagggttcagtccacgttgtgttttcacacctttctccaaggatcgaaccaaagatgtgtctccagtattgtgtctgtgcccctgtgtgtgttttgtggacagccgtgttgtctgactgtatccagctggcacttgacagggtgcagtcattggtgagaagaatcagaaaaagaagacccatcagcacaggttggataggggttagtgtaggagaccagttacagaatggcctggagtcttcaacttttgaccggtgcaggtgtgaccagagaccacctctgtggccacctcagatagtcatctcagtccaaggatcccaaaacacaaatctagaattgcaaagccagcctttatttccctggcaggcagccttgccagaaggcagagaggcagttctgaatcattctctcattcaccagtggtgacatccttcagtccctcacatctctgacagagcgggacagaatggatatttggctgaccttggtgagacctggagctgcctgtttcttccctaggggatcaccacggctctagggcattctaggatgaggtcagaccccttggccattggtgttattttttgtatagcttcagactgggttccagaacttaccattgaaaacagagcttttaggccaggtgtggtggctcacacctgtaatcccagcactttgggaggccgaggcaggtggatcgcttgagctcaggagttcgagagcagcctgggcaacatggtgaaagcccgtctctaccgaaaaatacaaaaaaaaatagctgggtgtggtggtacgtgcctgtagtcccagctacttcggggactgaggtgggaggatcacttgaacctaggaggtcgaggctgcagtgagccaagatcatgctactgcactccagcttaggtggcaaagtgagaccctgtctccaaaaaaagaaaatagagctttataagcagagagaagaaaataatcatctaagaccatctctccattcgacatgaagtgtaccttttctaaagacggtttccagctgcaacggctccactttgcaggcttgcagggtgtatacctgcgcattgggaacttgctggaacccctgatgcattttccttgagagcaggggtacttccgccttgccgttagcttgtggagaacgtgcttcttattcctggcaggcttcaagaacagctgcacatgtgccgctaactgaccgcgttgccattggcgacctggactctgaactcaggtttattctaaacccagtgagaggtgagggggagtgatgaaaggggatcagctgtatttgtgtgtgtgtgtgtgtgtgagcacctgacaaatctatgaaaccgagtgaaaggagaaatgttagattctttattattttattatatttatatggaaagctcgactctccctttggtaagtccgaagcatgttgtctgttcgaccgtgactgtcttcctcagtctgtgcctgtgattccagtcaccctgtagttactgacagaaattgactggactgtcattgtgtgaagtctaggaggaaatgtccattttaattgtatgatttggtcataagtaaggactatatttatgtcaccattattagatatatgtacttttgtaatgactgtgaaatacacttttccctcactacgactgcttctttatttgctgataaatcttaaaacaactaagtacgttgcaaataatagtacaggtaccatcttttatgtgaagttctttttctttttttgagacagggtcttgctctgtcacccaggctggagtgcagtggcacaatcacagttcactgcagcctcaacctctccagattcaagtgatcctcccatctcagcctcccaagtagctgggactacagttgtgcaccacaatgcctagctaattttttttgtattttgtagaaatggggtttcgccatgttgctcaggctgatctcaaactcctgggctccaacgatctgcccaccttggcctcccaaagtgctgagattacaggcgtgagccactgtgcctggccttatgtaaagctcttgacctagcatctgactggaaacaagcgggaattgcattggggggcattttctgggccagtttccctgccttattttacctgcaataggtgtgcctacaaaagtctttctgacatacacactgcaggtggcagttcaagaagaatcaacctttttcatttggggatattaggatcttccttggagactctgaaaatggcacacaaagaatggcagttatgaacgtgacttctagattttgtccttgtaggaggccttggtcatggataggggaagagggatgatgggatggaatgggagggtgggatctagatgacctcttgaaattgtcccagttactgcttccttcataactgttcacaaaacgtttttcatatataccaccttgtttcccctgggaagtgaggggacgaagccttagagggtgacatagctagaaagtggagaagctcactgttcactgtgcaaagccccagtagtcggattccggggaaaagaacaagtatctagttttgatgacaatgttttcttgtaagaatcccaacactttagaatagaaaaggacctaagattctctggctcagccccagcctttctgaagaagggaaaactagggacccagatggtttaaggacaccccagagtccccagatgagctggcatttgagctggtctggagcagagtttctccctcaaagaacacagagaaatcagagagtggctgccatggtcagagggggatgtcaacaacagatcaagccgtcatcacaacaggtatttctgaagttcctgcagggactaggtcttgcatttttaagtccttttcaaaactgtgcagcttcctgaaccttatgctgtttgtcccatccactcttgaagctggggaaagacagctttctttgggaactctgtctttctcagttgacttcccaagtaagcagcaaacccctggatagccttgttatctactttaggtattaagaggctattgggttttactagattaaatcaataaaacatttcctatagagccatatcatacactgaagcaaattcagaaagaatgcaaaactgctatcttttgggggagtctggaagcctgttggttagtgtatttattttctttgtggggtcttctgtgagctacaggcacagtaagaataattcagagcggtactgggagttggttcaatttcatgtcatcacttttcaatgaagggagtgcatttcctgagtagtaaatgagtatattatttgtggcagcttttctgttaagggagctttccagacatctggttgcaaagacaaataggatatatatttgtacttcttctctgtggacatgtttttgtgacacacagttatctctaggagttgacgtctgtgggcactaagggactgaggttgggggagaaagctgaggaggctttggaagagaaggtgaggagcatttcaggatttgcagtccccctccacatgtatccacatctgagctggtggtgttccaataatgaggcatttggggcaaccaattttggaaggatagaatagctttatgtggagcaaacttgaggatcaattggagttgagtggtttaaaacttaaagatggcacaggaagctgtatcatttacaggtgaaaaaaataaatgggtctgacttca

SEQ ID NO: 80 - DNA, 7219 bp, Homo sapiens
ggaccccggcaagcccgcgcacttggcaggagctgtagctaccgccgtccgcgcctccaaggtttcacggcttcctcagcagagactcgggctcgtccgccatgtccgccgcagacgaggttgacgggctgggcgtggcccggccgcactatggctctgtcctggataatgaaagacttactgcagaggagatggatgaaaggagacgtcagaacgtggcttatgagtacctttgtcatttggaagaagcgaagaggtggatggaagcatgcctaggggaagatctgcctcccaccacagaactggaggaggggcttaggaatggggtctaccttgccaaactggggaacttcttctctcccaaagtagtgtccctgaaaaaaatctatgatcgagaacagaccagatacaaggcgactggcctccactttagacacactgataatgtgattcagtggttgaatgccatggatgagattggattgcctaagattttttacccagaaactacagatatctatgatcgaaagaacatgccaagatgtatctactgtatccatgcactcagtttgtacctgttcaagctaggcctggcccctcagattcaagacctatatggaaaggttgacttcacagaagaagaaatcaacaacatgaagactgagttggagaagtatggcatccagatgcctgcctttagcaagattgggggcatcttggctaatgaactgtcagtggatgaagccgcattacatgctgctgttattgctattaatgaagctattgaccgtagaattccagccgacacatttgcagctttgaaaaatccgaatgccatgcttgtaaatcttgaagagcccttggcatccacttaccaggatatactttaccaggctaagcaggacaaaatgacaaatgctaaaaacaggacagaaaactcagagagagaaagagatgtttatgaggagctgctcacgcaagctgaaattcaaggcaatataaacaaagtcaatacattttctgcattagcaaatatcgacctggctttagaacaaggagatgcactggccttgttcagggctctgcagtcaccagccctggggcttcgaggactgcagcaacagaatagcgactggtacttgaagcagctcctgagtgataaacagcagaagagacagagtggtcagactgaccccctgcagaaggaggagctgcagtctggagtggatgctgcaaacagtgctgcccagcaatatcagagaagattggcagcagtagcactgattaatgctgcaatccagaagggtgttgctgagaagactgttttggaactgatgaatcccgaagcccagctgccccaggtgtatccatttgccgccgatctctatcagaaggagctggctaccctgcagcgacaaagtcctgaacataatctcacccacccagagctctctgtcgcagtggagatgttgtcatcggtggccctgatcaacagggcattggaatcaggagatgtgaatacagtgtggaagcaattgagcagttcagttactggtcttaccaatattgaggaagaaaactgtcagaggtatctcgatgagttgatgaaactgaaggctcaggcacatgcagagaataatgaattcattacatggaatgatatccaagcttgcgtggaccatgtgaacctggtggtgcaagaggaacatgagaggattttagccattggtttaattaatgaagccctggatgaaggtgatgcccaaaagactctgcaggccctacagattcctgcagctaaacttgagggagtccttgcagaagtggcccagcattaccaagacacgctgattagagcgaagagagagaaagcccaggaaatccaggatgagtcagctgtgttatggttggatgaaattcaaggtggaatctggcagtccaacaaagacacccaagaagcacagaagtttgccttaggaatctttgccattaatgaggcagtagaaagtggtgatgttggcaaaacactgagtgcccttcgctcccctgatgttggcttgtatggagtcatccctgagtgtggtgaaacttaccacagtgatcttgctgaagccaagaagaaaaaactggcagtaggagataataacagcaagtgggtgaagcactgggtaaaaggtggatattattattaccacaatctggagacccaggaaggaggatgggatgaacctccaaattttgtgcaaaattctatgcagctttctcgggaggagatccagagttctatctctggggtgactgccgcatataaccgagaacagctgtggctggccaatgaaggcctgatcaccaggctgcaggctcgctgccgtggatacttagttcgacaggaattccgatccaggatgaatttcctgaagaaacaaatccctgccatcacctgcattcagtcacagtggagaggatacaagcagaagaaggcatatcaagatcggttagcttacctgcgctcccacaaagatgaagttgtaaagattcagtccctggcaaggatgcaccaagctcgaaagcgctatcgagatcgcctgcagtacttccgggaccatataaatgacattatcaaaatccaggcttttattcgggcaaacaaagctcgggatgactacaagactctcatcaatgctgaggatcctcctatggttgtggtccgaaaatttgtccacctgctggaccaaagtgaccaggattttcaggaggagcttgaccttatgaagatgcgggaagaggttatcaccctcattcgttctaaccagcagctggagaatgacctcaatctcatggatatcaaaattggactgctagtgaaaaataagattacgttgcaggatgtggtttcccacagtaaaaaacttaccaaaaaaaataaggaacagttgtctgatatgatgatgataaataaacagaagggaggtctcaaggctttgagcaaggagaagagagagaagttggaagcttaccagcacctgttttatttattgcaaaccaatcccacctatctggccaagctcatttttcagatgccccagaacaagtccaccaagttcatggactctgtaatcttcacactctacaactacgcgtccaaccagcgagaggagtacctgctcctgcggctctttaagacagcactccaagaggaaatcaagtcgaaggtagatcagattcaagagattgtgacaggaaatcctacggttattaaaatggttgtaagtttcaaccgtggtgcccgtggccagaatgccctgagacagatcttggccccagtcgtgaaggaaattatggatgacaaatctctcaacatcaaaactgaccctgtggatatttacaaatcttgggttaatcagatggagtctcagacaggagaggcaagcaaactgccctatgatgtgacccctgagcaggcgctagctcatgaagaagtgaagacacggctagacagctccatcaggaacatgcgggctgtgacagacaagtttctctcagccattgtcagctctgtggacaaaatcccttatgggatgcgcttcattgccaaagtgctgaaggactcgttgcatgagaagttccctgatgctggtgaggatgagctgctgaagattattggtaacttgctttattatcgatacatgaatccagccattgttgctcctgatgcctttgacatcattgacctgtcagcaggaggccagcttaccacagaccaacgccgaaatctgggctccattgcaaaaatgcttcagcatgctgcttccaataagatgtttctgggagataatgcccacttaagcatcattaatgaatatctttcccagtcctaccagaaattcagacggtttttccaaactgcttgtgatgtcccagagcttcaggataaatttaatgtggatgagtactctgatttagtaaccctcaccaaaccagtaatctacatttccattggtgaaatcatcaacacccacactctcctgttggatcaccaggatgccattgctccggagcacaatgatccaatccacgaactgctggacgacctcggcgaggtgcccaccatcgagtccctgataggggaaagctctggcaatttaaatgacccaaataaggaggcactggctaagacggaagtgtctctcaccctgaccaacaagttcgacgtgcctggagatgagaatgcagaaatggatgctcgaaccatcttactgaatacaaaacgtttaattgtggatgtcatccggttccagccaggagagaccttgactgaaatcctagaaacaccagccaccagtgaacaggaagcagaacatcagagagccatgcagagacgtgctatccgtgatgccaaaacacctgacaagatgaaaaagtcaaaatctgtaaaggaagacagcaacctcactcttcaagagaagaaagagaagatccagacaggtttaaagaagctaacagagcttggaaccgtggacccaaagaacaaataccaggaactgatcaacgacattgccagggatattcggaatcagcggaggtaccgacagaggagaaaggccgaactagtgaaactgcaacagacatacgctgctctgaactctaaggccaccttttatggggagcaggtggattactataaaagctatatcaaaacctgcttggataacttagccagcaagggcaaagtctccaaaaagcctagggaaatgaaaggaaagaaaagcaaaaagatttctctgaaatatacagcagcaagactacatgaaaaaggagttcttctggaaattgaggacctgcaagtgaatcagtttaaaaatgttatatttgaaatcagtccaacagaagaagttggagacttcgaagtgaaagccaaattcatgggagttcaaatggagacttttatgttacattatcaggacctgctgcagctacagtatgaaggagttgcagtcatgaaattatttgatagagctaaagtaaatgtcaacctcctgatcttccttctcaacaaaaagttctacgggaagtaattgatcgtttgctgccagcccagaaggatgaaggaaagaagcacctcacagctcctttctaggtccttctttcctcattggaagcaaagacctagccaacaacagcacctcaatctgatacactcccgatgccacatttttaactcctctcgctctgatgggacatttgttacccttttttcatagtgaaattgtgtttcaggcttagtctgacctttctggtttcttcattttcttccattacttaggaaagagtggaaactccactaaaatttctctgtgttgttacagtcttagaggttgcagtactatattgtaagctttggtgtttgtttaattagcaatagggatggtaggattcaaatgtgtgtcatttagaagtggaagctattagcaccaatgacataaatacatacaagacacacaactaaaatgtcatgttattaacagttattaggttgtcatttaaaaataaagttcctttatatttctgtcccatcaggaaaactgaaggatatggggaatcattggttatcttccattgtgtttttctttatggacaggagctaatggaagtgacagtcatgttcaaaggaagcatttctagaaaaaaggagataatgtttttaaatttcattatcaaacttgggcaattctgtttgtgtaactccccgactagtggatgggagagtcccattgctaaaattcagctactcagataaattcagaatgggtcaaggcacctgcctgtttttgttggtgcacagagattgacttgattcagagagacaattcactccatccctatggcagaggaatgggttagccctaatgtagaatgtcattgtttttaaaactgttttatatcttaagagtgccttattaaagtatagatgtatgtcttaaaatgtgggtgataggaattttaaagatttatataatgcatcaaaagccttagaataagaaaagctttttttaaattgctttatctgtatatctgaactcttgaaacttatagctaaaacactaggatttatctgcagtgttcagggagataattctgcctttaattgtctaaaacaaaaacaaaaccagccaacctatgttacacgtgagattaaaaccaattttttccccattttttctccttttttctcttgctgcccacattgtgcctttattttatgagccccagttttctgggcttagtttaaaaaaaaaatcaagtctaaacattgcatttagaaagcttttgttcttggataaaaagtcatacactttaaaaaaaaaaaaaactttttccaggaaaatatattgaaatcatgctgctgagcctctattttctttctttgatgttttgattcagtattcttttatcataaatttttagcatttaaaaattcactgatgtacattaagccaataaactgctttaatgaataacaaactatgtagtgtgtccctattataaatgcattggagaagtatttttatgagactctttactcaggtgcatggttacagcccacagggaggcatggagtgccatggaaggattcgccactacccagaccttgttttttgttgtattttggaagacaggttttttaaagaaacattttcctcagattaaaagatgatgctattacaactagcattgcctcaaaaactgggaccaaccaaagtgtgtcaaccctgtttccttaaaagaggctatgaatcccaaaggccacatccaagacaggcaataatgagcagagtttacagctcctttaataaaatgtgtcagtaattttaaggtttatagttccctcaacacaattgctaatgcagaatagtgtaaaatgcgcttcaagaatgttgatgatgatgatatagaattgtggctttagtagcacagaggatgccccaacaaactcatggcgttgaaaccacacagttctcattactgttatttattagctgtagcattctctgtctcctctctctcctcctttgaccttctcctcgaccagccatcatgacatttaccatgaatttacttcctcccaagagtttggactgcccgtcagattgttgctgcacatagttgcctttgtatctctgtatgaaataaaaggtcatttgttcatgttaaaaaaaaa

SEQ ID NO: 81 - DNA, 1431 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctgggtaagttgaccatatatcctcacctcatggatattgaattggttatgatataaattggggatttgaagaagagtttctccttttgaccaaataaagtaccattagttga

SEQ ID NO: 82 - DNA, 6641 bp, Homo sapiens
gccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaacatcttgtaaaaaaaaaaaaaaaaaaaa

SEQ ID NO: 83 - DNA, 3555 bp, Homo sapiens
tttcccttttttttccttttggcaacccacacccttccacacacgctcaccccaaaattaaacaccaagatcctctaacttgtttggattgactgatgaagacataaagctctatgttttttgaggtggagtgagtggtttttcttcatttttaaatggccaaatgacagcttgacccagtttgctttccaatcaaagggcatttattttgaatgtctctttgtggcgcaagagccaacgcaaaaatgatggcggcttacaatggcggtacatctgcagcagcagcaggtcaccaccaccaccatcaccaccaccttccacacctccctcctcctcacctgcaccaccaccaccaccctcaacaccatcttcatccggggtcggctgccgctgtacaccctgtacagcagcacacctcttcggcagctgcggcagccgcagcagcggctgcagctgcagccatgttaaaccctgggcaacaacagccatatttcccatcaccggcaccggggcaggctcctggaccagctgcagcagccccagctcaggtacaggctgccgcagctgctacagttaaggcgcaccatcatcagcactcgcatcatccacagcagcagctggatattgagccggatagacctattggatatggagcctttggtgttgtctggtcagtaacagatccaagagatggaaagagagtagcgctcaaaaagatgcccaacgtcttccagaatctggtctcttgcaaaagggtcttccgggaattgaagatgttgtgtttttttaagcatgataatgtactctctgcccttgacatactccaacctccacacattgactattttgaagaaatatatgttgtcacagaattgatgcagagtgacctacataaaattatcgtctctcctcaaccactcagctcagatcatgtcaaagtttttctttatcagattttgcgaggtttgaaatatctccattcagctggcattttacatcgagacattaagccagggaatctccttgtgaacagcaactgtgttctaaagatttgtgattttggattggccagagtggaagaattagatgaatcccgtcatatgactcaggaagttgttactcagtattatcgggctccagaaatcctgatgggcagccgtcattacagcaatgctattgacatctggtctgtgggatgtatctttgcagaactactaggacgaagaatattgtttcaggcacagagtcccattcagcagttggatttgatcacggatctgttgggcacaccatcactggaagcaatgaggacagcttgtgaaggcgctaaggcacatatactcaggggtcctcataaacagccatctcttcctgtactctataccctgtctagccaggctacacatgaagctgttcatctcctttgcaggatgttggtctttgatccatccaaaagaatatccgctaaggatgccttagcccacccctacctagatgaagggcgactacgatatcacacatgtatgtgtaaatgttgcttttccacctccactggaagagtttataccagtgactttgagcctgtcaccaatcccaaatttgatgacactttcgagaagaacctcagttctgtccgacaggttaaagaaattattcatcagttcattttggaacagcagaaaggaaacagagtgcctctctgcatcaaccctcagtctgctgcttttaagagctttattagttccactgttgctcagccatctgagatgcccccatctcctctggtgtgggagtgatggtggaagataatgtactactgaagatgtaatgtagctttccactggagtctgggatttgcaattctggaggttaatcatgcttgtactgtaattttactaatgaagttttaaattaacaaccactacttgtatgatatgaataatatttagaaatgttactagacttttaatcttgtaaagtggttgtgcttttagaagaaaaatattttacccagagttgcacatgttttatgaatttagtgcagctgttatggctcacctcagaacaaaagagaattgaaccaaatttgggagtttggggttttatgttttgtttttcttttctaaaatgaagtgagattgttcacacacacacacacacacacacacacacacaaacacaaaggacagtcatacattttgatatttgagccattcctaaagatttggggttttctaaaactaaagaatctaggaaccttgcctgcgaccaatcatggagccacgtgagctgatcgtggctgcacctggggggagggtagggaggaggggcatgccacctaatgatcaagccctataattagcttctcattagagccgtgatggtgatgtgtgctgtctaaaatccaatgttgtgggtagagagaatgagtttgtgactaggagagactaaacttttgttttccttacccagtataaatatatatatatatatttaatctatttttattagaagtttttctgctctttcttacataaaagaaccccaagcatgcatctttcatgtgtgtaaataattcatttctgggctaatttcaaaagaatcccaatattgctgtatagaaagagaactagcttgcacattttaggtctgtgaaattttgtgagacttttcctgcactggacagtaaaaaaataataaaagacaaaaacaaatttaaaaaaaaatttaagccacaaaaaaaggcacatagggaattatgtcaaatgtgtttgtgtccttaggcaaagctgtgggagtcttgaaatgtcacagtagtaaataacttattactttttgacaagttctttttttctgttggaacactgaattcttctgtgcatatgtacatatgaatacaaatcgaaggcctctacctcctggagttatacaacttggcttgtttacctccacatgctgatgatgactattttttttttttgagttcagtgtggagacttgaaacttgaatgtcccttccaacttttatattaaaaataaaaagacaagaaaattgaagatcatttttcacctgtacaaggaatactaatggatcgtcttaaaattggtctaggaaaaaaccttggtgtgtgttatggacaattaactgttaaagttattgtaagttatctgtatttagcagagtattttcaacttgagtgatctgagctgaatttgaagactattaataagttatgtttggaagttttaacttcaatgaagtaattatttgctgtgaaagaaacaaacattgaattactaaacaaagatggtgcaatatctttgttttttttttatgaggctcctgagaatcaacccaactgaagcatttcaattcacttgaatgagaaacgtgtttagtatcaaaagagcccaagaagacactggtgtgaaaggtacaatctcagaggttggtcaattaccgtggcacactttctggtcactttgtacaatgtagatttgaagtacagtggtgaaaacattaaatgtgacatttgaaaaaaaaaaaaaaaaaaa

SEQ ID NO: 84 - DNA, 2962 bp, Homo sapiens
acatttctgcagccgcgcggcgagccattcgcggcggctgctgcagctcctactgcatcttccttctcttcctttcctcgggctccggtctcggagtcggagagcgcgcctcgcttccagagcccccggacccggcgagtcagcgatcgccgagccggccaccatgcccggcagaccgcgccactaggcgctcctcgcggctcccacccggcggcggcggcggcggcggcggcgtccgcgatggtttcagacgctgaaggattttgcatctgatcgctcggcgtttcaaagaagcagcgatcggagatggatgtctctctttgcccagccaagtgtagtttctggcggattttcttgctgggaagcgtctggctggactatgtgggctccgtgctggcttgccctgcaaattgtgtctgcagcaagactgagatcaattgccggcggccggacgatgggaacctcttccccctcctggaagggcaggattcagggaacagcaatgggaacgccagtatcaacatcacggacatctcaaggaatatcacttccatacacatagagaactggcgcagtcttcacacgctcaacgccgtggacatggagctctacaccggacttcaaaagctgaccatcaagaactcaggacttcggagcattcagcccagagcctttgccaagaacccccatttgcgttatataaacctgtcaagtaaccggctcaccacactctcgtggcagctcttccagacgctgagtcttcgggaattgcagttggagcagaactttttcaactgcagctgtgacatccgctggatgcagctctggcaggagcagggggaggccaagctcaacagccagaacctctactgcatcaacgctgatggctcccagcttcctctcttccgcatgaacatcagtcagtgtgaccttcctgagatcagcgtgagccacgtcaacctgaccgtacgagagggtgacaatgctgttatcacttgcaatggctctggatcaccccttcctgatgtggactggatagtcactgggctgcagtccatcaacactcaccagaccaatctgaactggaccaatgttcatgccatcaacttgacgctggtgaatgtgacgagtgaggacaatggcttcaccctgacgtgcattgcagagaacgtggtgggcatgagcaatgccagtgttgccctcactgtctactatcccccacgtgtggtgagcctggaggagcctgagctgcgcctggagcactgcatcgagtttgtggtgcgtggcaaccccccaccaacgctgcactggctgcacaatgggcagcctctgcgggagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacagggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctagtggtggctggtggtcatgaattcatactctgttgcctcctctctccctgcctcacatctcccttccacctcacaactccttccatccttgactgaagcgaacatcttcatataaactcaagtgcctgctacacatacaacactgaaaaaaggaaaaaaaaagaaagaaaaaaaaaccc

SEQ ID NO: 85 - DNA, 4991 bp, Homo sapiens
ggcggcgcgcaagggacgtgcggagtgagtggcgctgcgggtggggccgtcggcggcgctggtgagctttgcggagctgggcggtgccgaggaggaggaggtggcggcctgggtctgacgcggccctgttcgagggggcctctcttgtttatttatttattttccgtgggtgcctccgagtgtgcgcgcgctctcgctacccggcggggagggggtggggggagggcccgggaaaagggggagttggagccggggtcgaaacgccgcgtgacttgtaggtgagagaacgccgagccgtcgccgcagcctccgccgccgagaagcccttgttcccgctgctgggaaggagagtctgtgccgacaagatggcggacggggagctgaacgtggacagcctcatcacccggctgctggaggtacgaggatgtcgtccaggaaagattgtgcagatgactgaagcagaagttcgaggcttatgtatcaagtctcgggagatctttctcagccagcctattcttttggaattggaagcaccgctgaaaatttgtggagatattcatggacaatatacagatttactgagattatttgaatatggaggtttcccaccagaagccaactatcttttcttaggagattatgtggacagaggaaagcagtctttggaaaccatttgtttgctattggcttataaaatcaaatatccagagaacttctttctcttaagaggaaaccatgagtgtgctagcatcaatcgcatttatggattctatgatgaatgcaaacgaagatttaatattaaattgtggaagaccttcactgattgttttaactgtctgcctatagcagccattgtggatgagaagatcttctgttgtcatggaggattgtcaccagacctgcaatctatggagcagattcggagaattatgagacctactgatgtccctgatacaggtttgctctgtgatttgctatggtctgatccagataaggatgtgcaaggctggggagaaaatgatcgtggtgtttcctttacttttggagctgatgtagtcagtaaatttctgaatcgtcatgatttagatttgatttgtcgagctcatcaggtggtggaagatggatatgaattttttgctaaacgacagttggtaaccttattttcagccccaaattactgtggcgagtttgataatgctggtggaatgatgagtgtggatgaaactttgatgtgttcatttcagatattgaaaccatctgaaaagaaagctaaataccagtatggtggactgaattctggacgtcctgtcactccacctcgaacagctaatccgccgaagaaaaggtgaagaaaggaattctgtaaagaaaccatcagatttgttaaggacatacttcataatatataagtgtgcactgtaaaaccatccagccatttgacaccctttatgatgtcacacctttaacttaaggagacgggtaaaggatcttaaatttttttctaatagaaagatgtgctacactgtattgtaataagtatactctgttatagtcaacaaagttaaatccaaattcaaaattatccattaaagttacatcttcatgtatcacaatttttaaagttgaaaagcatcccagttaaactagatgtgatagttaaaccagatgaaagcatgatgatccatctgtgtaatgtggttttagtgttgcttggttgtttaattattttgagcttgttttgtttttgtttgttttcactagaataatggcaaatacttctaatttttttccctaaacatttttaaaagtgaaatatgggaagagctttacagacattcaccaactattattttcccttgtttatctacttagatatctgtttaatcttactaagaaaactttcgcctcattacattaaaaaggaattttagagattgattgttttaaaaaaaaatacgcacattgtccaatccagtgattttaatcatacagtttgactgggcaaactttacagctgatagtgaatattttgctttatacaggaattgacactgatttggatttgtgcactctaatttttaacttattgatgctctattgtgcagtagcatttcatttaagataaggctcatatagtattacccaactagttggtaatgtgattatgtggtaccttggctttaggttttcattcgcacggaacaccttttggcatgcttaacttcctggtaacaccttcacctgcattggttttctttttcttttttctttctttttttttttttttttttttttgagttgttgtttgtttttagatccacagtacatgagaatccttttttgacaagccttggaaagctgacactgtctctttttcctccctctatacgaaggatgtatttaaatgaatgctggtcagtgggacattttgtcaactatgggtattgggtgcttaactgtctaatattgccatgtgaatgttgtatacgattgtaaggcttatgtcactaaagatttttattctgattttttcataatcaaaggtcatatgatactgtatagacaagctttgtagtgaagtatagtagcaataatttctgtacctgatcaagtttattgcagcctttcttttcctatttcttttttttaagggttagtattaacaaatggcaatgagtagaaaagttaacatgaagattttagaaggagagaacttacaggacacagatttgtgattctttgactgtgacactattggatgtgattctaaaagcttttattgagcattgtcaaatttgtaagcttcatagggatggacatcatatctataatgcccttctatatgtgctaccatagatgtgacatttttgaccttaatatcgtctttgaaaatgttaaattgagaaacctgttaacttacattttatgaattggcacattgtattacttactgcaagagatatttcattttcagcacagtgcaaaagttctttaaaatgcatatgtctttttttctaattccgttttgttttaaagcacattttaaatgtagttttctcatttagtaaaagttgtctaattgatatgaagcctgactgattttttttttccttacagtgagacatttaagcacacattttattcacatagatactatgtccttgacatattgaaatgattcttttctgaaagtattcatgatctgcatatgatgtattaggttaggtcacaaaggttttatctgaggtgatttaaataacttcctgattggagtgtgtaagctgagcgatttctaataaaattttagttgtacacttttagtagtcatagtgaagcaggtctagaaaataagcctttggcagggaaaaagggcaatgttgattaatctcagtattaaaccacattaatctgtatcccattgtctggcttttgtaaattcatccaggtcaagactaagtatgttggttaataggaatccttttttttttttttaaagactaaatgtgaaaaaataatcactacttaagctaattaatattggtcattaaatttaaaggatggaaatttatcatgtttaaaaattattcaagcactcttaaaaccacttaaacagcctccagtcataaaaatgtgttctttacaaatatttgcttggcaacacgacttgaaataaataaaactttgtttcttaggagaaaatgattctgtaattccagtgtcactaatttatattgttctttcctctgatttttttcaggttagtgatttttttgtatacaatttaatccaaatgttatgacattcagaaatcatgaaacacagtagatatctgttataatgtggtgtatcacatggattataaagcaaagttatggtcgatttctattcttgaaagaatcaactacagtgaatcctttgcatttgaagccttaacatgcattgctttaattttgcccagggacaaattttaataatcagcaagactggtttgtgcaaagcgttgagtcatcaggtatttagagcctagccagctacccagtatccatgctgccatatcccttcattgtaaaaagtacctaaacattcgtgaaatgattttttttagctgaaaaatgctggcaagaagaattttaaagcttaaaataggtggtaaatttgaagtatgagtgtgttcacgagaaacataggcttttcaaaaaaatttttattcaaggcaaagcaaggaacatcttgagatatgtctcaagaatataaagatgtattattttaagccaaggagctgaaatatatctcagtttataaattcaggtatattctttttgtctccatggcaaccataacttttgaaccaaaaaaaattgtttttacatctttatgctgaaaatgtgtttagattaggaatatggtcgggctgaatttgctgttgctccctaaccaaatccacctcttgttttccttgtgagtccatggctaaatcaaagctgcccctgagaagagacttaatccaagcctgattgtactagtggcatcacttagaagtaggctttccctcttcctagtagatctcaatgttttataattccttaaaacagctgaaaattgggacaacatactttacgcaatgaacagtagttaaataggaaataaactagttccatataagtatacacctagagttttaattacctttataatgtttcttaaaagtgaaacttagatacaattgtgattggatacttagatactaagtgaaacttagtgtaacaattttgatctgttaaattggattttacatgtacatttgaatgccagaatttctaaataaatcccctggttaggaaattttaaaagtcaaagcttgttttcttcaaccactaccttctacattggttgacttagaccgtaagctttttaagtttctcattgtaatttaccttctcatgcagattgctgatgttttattaaaccttatttttacaaaaatgaaaaaa

SEQ ID NO: 86 - DNA, 1828 bp, Homo sapiens
gcggccgcatgagccgcgcgcgtggggcgctgtgccgggcctgcctcgcgctggccgcggccctggccgcgctgctgttactgccgctgccgctgccccgcgcgcccgccccggcccggacccccgccccggccccgcgcgcgcccccgtcccggcccgctgcccccagcctgcggcctgacgacgtcttcatcgccgtcaagaccacccggaagaaccacgggccgcgcctgcggctgctgctgcgcacctggatctcccgggcccgccagcagacgtttatcttcaccgacggggacgaccctgagctcgagctccagggcggcgaccgtgtcatcaacaccaactgctcggcggtgcgcactcgtcaggccctctgctgcaagatgtccgtggagtatgacaagttcattgagtccgggcgcaagtggttttgccacgtggatgatgacaattatgtgaacgccaggagcctcctgcacctgctctccagcttctcacccagccaggacgtctacctggggcggcccagcctggaccaccccattgaggccaccgagagggtccagggtggcagaactgtgaccacggtcaagttctggtttgctactggtggggccgggttctgcctcagcagaggccttgccctcaagatgagcccatgggccagcctgggcagcttcatgagcacagctgagcaggtgcggctgccggatgactgcacagttggctacatcgtggaggggctcctgggcgcccgcctgctgcacagccccctcttccactctcacctggagaacctgcagaggctgccgcccgacaccctgctccagcaggttaccttgagccatgggggtcctgagaacccacataacgtggtgaacgtggctggaggcttcagcctgcatcaagaccccacacggtttaagtctatccattgtcttctgtacccagacacggactggtgtcccaggcagaaacagggcgccccgacctctcggtgacaccaaccaccccgacccagggctgcctggctctgtcccaggcgcggggaaccagagccccctatgggctcagtgggctccctcaggtgccacggccacaccagtgagatgcaggcacctggcagaccctctggctagcctgcagccccccctctcccagcccctggtgggctgcggtgatgggtgttttgggagaacgaagacagccaggctgatggccagggccgcagtgcccctccccccgacccagccccaaggttgatcccacgggaacaggcttccaccccagcacttgcgcacctgggagggagctgccatccgggctccattacctgttgctgaaggcgggtgctaggctggctgggtgtcaaggagcaggctccaggccaaggtcctggcccagccacggccattgcaagggctcagcctggcaggctttgtgggggacgccgccctctctgccgcaggctgggtgcacggccgggcaccacagtgggactcaggcccgggaaggtcatgttctcgaccagagctttgctcccagtccaggcgcctcattcggaggcctcttgactgggaccacagagatgttttctccgctctgacttgtggctcaggactactttctgggtcgtgctcctgccccactgtgcctgggcccataaacaacaggagccttttgttccgctgacctgcccatcccagcagacccacctccccgcctggtgccttccatggagggaaatgggacaggggccgtcatctcccctctgcctcctgctttgctgttgccgagcacgagtaaagcttttgttcttacttca

SEQ ID NO: 87 - DNA, 1955 bp, Homo sapiens
cccggctccacccccaagccaggcgaggcaggttccgaggttggaacacctggcgagtcctcggtgtcggtggccggcagtcatctcgcggccgttcaggataagccagagacatgggaaaaccaatggaagtgtttcaaagttcttctctttaatcacttctgtatccagctgcctttgatttgtggaacctattattttacagagtatttcaatattccttatgattgggaaagaatgccaagatggtattttcttttggcaagatgctttggttgtgcagtcattgaagatacttggcactattttctgcatagactcttacaccacaaaagaatatacaagtatattcataaagttcatcatgagtttcaggctccatttggaatggaagctgaatatgcacatcctttggagactctaattcttggaactggatttttcattggaatcgtgcttttgtgtgatcatgtaattcttctttgggcatgggtgaccattcgtttattagaaactattgatgtccatagtggttatgatattcctctcaaccctttaaatctgatccctttctatgctggttctcggcatcatgatttccaccacatgaacttcattggaaactatgcttcaacatttacatggtgggatcgaatttttggaacagactctcagtataatgcctataatgaaaagaggaagaagtttgagaaaaagactgaataaatatctcacgtaaaccttcctgaaagataaacgttttcctgaattcagaaactagtagctaacattgcttctggagagcagaaataagcatgtcttctggctactaagtgataaaaagaacattaacaacctttaattaccttcctagtgggaactttttctactttacctacaagttctatatatgtagaaatgaataaatatatatttaagtacagttttcatgaggaagttttaaaagaccatgttcctaagcttccaagaaggttttggatactagaagtattaatctatggcttttctcccagtaaaaccataggcctgaagttcacattgggtctttaaatcttttagatatatactggtcatttcagaaaattcttcatagtggtattggccttatatttaactttttttttattttttttttgagacaaagccacactctgtctccttggctggagtgtggtggcacagtctcagctcactgcaacctctgcctcccagttcaagcaattcttctgcctcagcctcccaagtagctgggattacaggcacccgccaccacgcccagctaatttttgtatttttgtagagatggggtttcacgatgttggccaggctggtctcaaacttctgacctcaagtgatctgcccaccttggcctcccaaagtgctgggattacaggtgtaagccactgcgcccggcctttttaactttaaacatgttttagaattcacctaaagatcaaaatatcatggattgaacctcatcaattgatagcagtgagtgactgaagcttccaaatcaagaaaagccggcaccaagaacttccattctaatctagagctgaccagtttgagctgattctctctttgaagagtccttcttgattgcagtgcagtactggcatttctgaatggatgtaagtggagtattttagtctaaaggcttttcaaattacttgaatttttttaaaaattgaggagctttatttctatttacccttccatttttgtatatcaaatttccattgtcattaaaaactgtatcttgaaactttgtgaactgacttgctgtatttgcactttgagctcttgaaataaatgtgatttttgtgtgattatctggtttccagttttaaacattaactgtcaccttttattcttaaacttgaaagtacagaaatcattaaattattaagttgtacaataaaaaaaaaaaaaaa

SEQ ID NO: 88 - DNA, 1451 bp, Homo sapiens
actgcctggaaacgggctgggcctgcctcggacgccgccggtgtcgcggattctctttccgcccgctccatggcggtggatgcctgactggaagcccgagtgggatgcggctgacgcggaagcggctctgctcgtttcttatcgccctgtactgcctattctccctctacgctgcctaccacgtcttcttcgggcgccgccgccaggcgccggccgggtccccgcggggcctcaggaagggggcggcccccgcgcgggagagacgcggccgagaacagtccactttggaaagtgaagaatggaatccttgggaaggagatgaaaaaaatgagcaacaacacagatttaaaactagccttcaaatattagataaatccacgaaaggaaaaacagatctcagtgtacaaatctggggcaaagctgccattggcttgtatctctgggagcatatttttgaaggcttacttgatcccagcgatgtgactgctcaatggagagaaggaaagtcaatcgtaggaagaacacagtacagcttcatcactggtccagctgtaataccagggtacttctccgttgatgtgaataatgtggtactcattttaaatggaagagaaaaagcaaagatcttttatgccacccagtggttactttatgcacaaaatttagtgcaaattcaaaaactccagcatcttgctgttgttttgctcggaaatgaacattgtgataatgagtggataaacccattcctcaaaagaaatggaggcttcgtggagctgcttttcataatatatgacagcccctggattaatgacgtggatgtttttcagtggcctttaggagtagcaacatacaggaattttcctgtggtggaggcaagttggtcaatgctgcatgatgagaggccatatttatgtaatttcttaggaacgatttatgaaaattcatccagacaggcactaatgaacattttgaaaaaagatgggaacgataagctttgttgggtttcagcaagagaacactggcagcctcaggaaacaaatgaaagtcttaagaattaccaagatgccttgcttcagagtgatctcacattgtgcccggtcggagtaaacacagaatgctatcgaatctatgaggcttgctcctatggctccattcctgtggtggaagacgtgatgacagctggcaactgtgggaatacatctgtgcaccacggtgctcctctgcagttactcaagtccatgggtgctccctttatctttatcaagaactggaaggaactccctgctgttttagaaaaagagaaaactataattttacaagaaaaaattgaaagaagaaaaatgttacttcagtggtatcagcacttcaagacagagcttaaaatgaaatttactaatattttagaaagctcatttttaatgaataataaaagttaattatctttttgagct

SEQ ID NO: 89 - DNA, 829 bp, Homo sapiens
ccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgttaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagag

SEQ ID NO: 90 - DNA, 2780 bp, Homo sapiens
gtgggcggaccgcgcggctggaggtgtgaggatccgaacccaggggtggggggtggaggcggctcctgcgatcgaaggggacttgagactcaccggccgcacgccatgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaaattatactctcaccatttggatcctgtgtggagagggaatgtgaaatttacatcatttctttttgggagagacttgttttggatgccccctaatccccttctcccctgcactgtaaaatgtgggattatgggtcacaggaaaaagtgggttttttagttgaattttttttaacattcctcatgaatgtaaatttgtactatttaactgactattcttgatgtaaaatcttgtcatgtgtataaaaataaaaaagatcccaaat

SEQ ID NO: 91 - DNA, 2029 bp, Homo sapiens
ggcggcgggcggcgggcggcggggaccgggtgcggtggtggctgcggcggcggcggcgggagcagcatggattggggcactgagctgtgggatcagttcgaggtgctcgagcgccacacgcagtgggggctggacctgttggacagatatgtaaagttcgtgaaagaacgcaccgaagtggaacaggcttacgccaaacaactgcggagcctggtgaaaaaatatctgcccaagagacctgccaaggatgatcctgagtccaaattcagccagcaacagtccttcgtacagattctccaggaggtgaatgactttgcaggccagcgggagctggtggctgagaacctcagtgtccgtgtatgtcttgagctgaccaagtactcacaagagatgaaacaggagaggaagatgcacttccaagaagggcggcgggcccagcagcagctggaaaatggctttaaacagctggagaatagtaagcgtaaatttgagcgggactgccgggaggcagagaaggcagcccagactgctgaacggctagaccaggatatcaacgccaccaaggctgatgtggagaaggccaagcagcaagcccaccttcggagtcacatggccgaagaaagcaaaaacgaatatgcggctcaactgcagcgcttcaaccgagaccaagcccacttctatttttcacagatgccccagatattcgataagctccaagacatggatgaacgcagggccacccgcctgggtgccgggtatgggctcctgtcggaggccgagctggaggtggtgcccataatagccaagtgcttggagggcatgaaggtggctgcaaatgctgtggatcccaagaacgactcccacgtccttatagagctgcacaagtcaggttttgcccgcccgggcgacgtggaattcgaggacttcagccagcccatgaaccgtgcaccctccgacagcagtctgggcaccccctcggatggacggcctgaactccgaggcccgggtcgcagccgcaccaagcgctggccttttggcaagaagaacaagacagtggtgaccgaggattttagccacttgcccccagagcagcagcgaaaacggcttcaacagcagttggaagaacgcagtcgtgaacttcagaaggaggttgaccagagggaagccctaaagaaaatgaaggatgtctatgagaagacacctcagatgggggaccccgccagcttggagccccagatcgctgaaaccctgagcaacattgaacggctgaaattggaagtgcagaagtatgaggcgtggctggcagaagctgaaagtcgagtccttagcaaccggggagacagcctgagccggcacgcccggcctcccgacccccccgctagcgccccgccagacagcagcagcaacagcgcatcacaggacaccaaggagagctctgaagagcctccctcagaagagagccaggacacccccatttacacggagtttgatgaggatttcgaggaggaacccacatcccccataggtcactgtgtggccatctaccactttgaagggtccagcgagggcactatctctatggccgagggtgaagacctcagtcttatggaagaagacaaaggggacggctggacccgggtcaggcggaaagagggaggcgagggctacgtgcccacctcctacctccgagtcacgctcaattgaaccctgccagagacgggaagaggggggctgtcggctgctgcttctgggccacggggagccccaggacctatgcactttatttctgaccccgtggcttcggctgagacctgtgtaacctgctgccccctccacccccaacccagtcctacctgtcacaccggacggacccgctgtgccttctaccatcgttccaccattgatgtacatactcatgttttacatcttttctttctgccgctcggctccggccattttgttttatacaaaaatgggaaaaaaaaaaaagaaattatataaagttcctagaaaaaaaaaaaaaaaaaaa

SEQ ID NO: 92 - DNA, 1235 bp, Homo sapiens
ctcagctccgcaccgcaactgaagatctgccgccgcggaacagttgcgtctccatctggctaccaacccacccaagctttcttctccaccaccaccaccttccttccttccccctcctccccctcctttccgtcttccctctccacccccgcccccaatctcctcctttttttctcactacgagcggttgctgatgctgaagccgagcgtcacttcggctcccacggcagacatggcgacattgacagtggtccagccgctcaccctggacagagatgttgcaagagcaattgaattactggaaaaactacaggaatctggagaagtaccagtgcacaagctacaatccctcaaaaaagtgcttcagagtgagttttgtacagctattcgagaggtgtatcaatatatgcatgaaacgataactgttaatggctgtcccgaattccgtgcgagggcaacagcaaaggcaacagttgcagcttttgcagctagtgaaggccactcccaccctcgagtagttgaactgccaaagactgatgaaggccttggttttaatgtgatgggaggaaaggagcaaaattcccccatttatatctctcgcataattcctggaggggtggctgaaagacacggaggcctcaaaagaggagaccagctgctatcagtgaacggagtgagtgtggaaggagaacaccatgagaaagctgtggaactactcaaggctgctaaagacagcgtcaagctggtggtgcgatacaccccaaaagttctggaagaaatggaggctcgctttgaaaagctacgaacagccaggcgtcggcagcagcagcaattgctaattcagcagcagcaacagcagcagcagcaacaaacacaacaaaaccacatgtcataggcccttgagggaaagctacttgatcaaacatccgatagtcacaaatttgaaaccgtgcttcagaatcccagcacatagtaaaagacaacactgataattatacctgtcaagaagctgtgaacacatggtgtataaattctttaccaaggcaactcaacaccttctttctctgggcttgaaccgccactgctcacgtgggctttacatacattgaccttccattcactgcagtgggaattctcagtgtgcagagggagaggttttctagtctgcaaactgaaacagtgtaagaagaataaagtctatgacttttaaataaatcaccagggccaagaaaaag